(19) United States
(12) Patent Application Publication
(10) Pub. No.: US 2022/0323648 A1
(43) Pub. Date: Oct. 13, 2022

(54) EXTRACELLULAR MATRIX DEVICES AND METHODS OF MANUFACTURE

(71) Applicants: University of Pittsburgh - Of the Commonwealth System of Higher Education, Pittsburgh, PA (US); RENERVA, LLC, Pittsburgh, PA (US)

(72) Inventors: Lorenzo Soletti, Pittsburgh, PA (US); Bryan Brown, Pittsburgh, PA (US); Josh Bowman, Pittsburgh, PA (US); Nicole Cwalina, Roswell, GA (US); Brandon Burger, Pittsburgh, PA (US); J. Christopher Flaherty, Nottingham, NH (US)

(21) Appl. No.: 17/762,858

(22) PCT Filed: Sep. 30, 2020
(86) PCT No.: PCT/US2020/053570

§ 371 Date: Mar. 23, 2022

Related U.S. Application Data
(60) Provisional application No. 62/907,784, filed on Sep. 30, 2019.

(51) Int. Cl. A61L 27/36

(52) U.S. Cl. CPC A61L 27/3633; A61L 27/3683

(57) ABSTRACT

Systems, devices, and methods for treating a nerve injury in a patient are provided herein. The system includes an extracellular matrix, a neutralizing element, and a reconstituting element. The extracellular matrix is configured to promote and/or sustain the growth of tissue and/or associated tissue properties proximate the nerve injury.

### RELATED APPLICATIONS

This application claims benefit to U.S. Provisional Patent Application Ser. No. 62/907,784 (Client Docket No. REN-005-PR1), entitled “Extracellular Matrix Devices and Methods of Manufacture”, filed Sep. 30, 2019, the content of which is incorporated herein by reference in its entirety for all purposes.

This application claims benefit to U.S. Provisional Patent Application Ser. No. 62/954,813 (Client Docket No. REN-004-PR1), entitled “Extracellular Matrix Systems, Devices and Methods of Deployment”, filed Dec. 30, 2019, the content of which is incorporated herein by reference in its entirety for all purposes.

This application is related to:
U.S. Pat. No. 8,361,503, Issued Jan. 29, 2013;
U.S. Pat. No. 8,691,276, Issued Apr. 8, 2014;
U.S. Pat. No. 9,737,635, Issued Aug. 22, 2017;
U.S. patent Ser. No. 10/004,827, Issued Jun. 26, 2018;
U.S. patent Ser. No. 10/179,192, Issued Jan. 15, 2019;
U.S. patent Ser. No. 10/213,526, Issued Feb. 26, 2019;
U.S. patent Ser. No. 10/729,813, Issued Aug. 4, 2020; and
U.S. patent Ser. No. 10/772,989, Issued Sep. 15, 2020;

the content of each being incorporated by reference herein, in its entirety.

This application is related to U.S. patent application Ser. No. 15/996,916 (Client Docket No. REN-001-US-CON2), entitled “Extracellular Matrix-Derived Gels and Related Methods”, filed Jun. 4, 2018 published as US 2019/0038803; U.S. patent application Ser. No. 16/208,196 (Client Docket No. REN-002-US-CON2), entitled “Injectable Peripheral Nerve Specific Hydrogel”, filed Dec. 3, 2018; U.S. patent application Ser. No. 16/238,826 (Client Docket No. REN-003-US-CON1), entitled “Methods for Preparation of a Terminally Sterilized Hydrogel Derived from Extracellular Matrix”, filed Jan. 3, 2019, published as US 2019/0374683; and U.S. patent application Ser. No. 16/288,831 (Client Docket No. REN-001-US-CON3), entitled “Extracellular Matrix-Derived Gels and Related Methods”, filed Feb. 28, 2019 published as US 2019/0201581; the content of each of which is incorporated herein by reference in its entirety for all purposes.

### FIELD OF THE INVENTION

The present inventive concepts relate generally to improved nerve injury treatment systems, devices and methods.

### BACKGROUND

Peripheral Nerve Injury (PNI) can severely impact the quality of life, productivity, and interpersonal relationships of injured patients. For example, nerve injuries in the upper extremities (i.e., hand, wrist, elbow, or shoulder) can prevent patients from being able to perform basic daily activities such as getting dressed, working, or feeding themselves. Facial nerve injuries impede vocalization and are associated with social stigma and withdrawal. Injuries in the lower limbs can prevent patients from having a normal gait; in some severe cases, patients have reported preferring leg amputation and replacement with a functional modern leg prosthesis. Existing FDA-approved nerve products are primarily indicated for use as passive support or to prevent complications (e.g. mechanical instability, neuroma, or donor site morbidity associated with autograft). None of these products has shown meaningful clinical improvement in functional outcomes.

Surgeons performing nerve repair often give their patients very poor prognoses and little hope. Nerve regeneration typically requires 3-18 months to complete and satisfactory terminal functional recovery is often less than 50%. Each year in the U.S. alone, surgeons perform around 550,000 procedures to repair peripheral nerves affected by traumatic or iatrogenic nerve injury.

There is a need for improved nerve injury treatment systems, devices and methods.

### SUMMARY

According to an aspect of the present inventive concepts, a system for treating a patient comprising: an extracellular matrix comprising tissue harvested from a tissue source; a neutralizing element; and a reconstituting element. The system can be configured to provide a therapeutic benefit to the patient.

In some embodiments, the extracellular matrix comprises a concentration of native protein between 5 mg/mL and 50 mg/mL. The extracellular matrix can comprise a concentration of native protein between 10 mg/mL and 30 mg/mL. The extracellular matrix can comprise a concentration of native protein of 20 mg/mL. The concentration of native protein can be configured to improve a parameter of the extracellular matrix, and the parameter can be selected from the group consisting of: solubility; reconstitution; mechanical modulus; in vivo remodeling; durability; and combinations thereof.

In some embodiments, the neutralizing element and/or reconstituting element are configured to interact with the extracellular matrix, and the interaction causes a change to the extracellular matrix. The interaction can cause a physical change to the extracellular matrix. The interaction can cause a chemical change to the extracellular matrix. The neutralizing element can be configured to counteract a property of the extracellular matrix, and the property can be selected from the group consisting of: physical; mechanical; chemical; biological; and combinations thereof. The extracellular matrix can comprise a fluid and the neutralizing element can comprise a phosphate-buffered saline (PBS) solution. The neutralizing element can comprise a phosphate-buffered saline (PBS) solution comprising a concentration configured to modify a mechanical strength of the extracellular matrix. The neutralizing element can comprise a phosphate-buffered saline (PBS) solution comprising a concentration configured to modify a gelation time of the extracellular matrix. The neutralizing element can comprise a phosphate-buffered saline (PBS) solution comprising a concentration configured to modify a gelation temperature of the extracellular matrix. The reconstituting element can be configured to modify a property of the extracellular matrix, and the property can be selected from the group consisting of: physical; mechanical; chemical; biological; and combinations thereof.

In some embodiments, the neutralizing element is configured to interact with the reconstituting element, and the interaction causes a change to the reconstituting element. The neutralizing element can be configured to counteract a property of the reconstituting element, and the property can be selected from the group consisting of: physical; mechanical; chemical; biological; and combinations thereof.

In some embodiments, the neutralizing element comprises a solution comprising sodium hydroxide (NaOH), phosphate-buffered saline (PBS), and/or water. The sodium hydroxide can comprise a molar concentration of 0.2 M. The phosphate-buffered saline (PBS) can comprise a concentration of between 0.5× and 1.0×.

In some embodiments, the reconstituting element is configured to interact with the neutralizing element, and the interaction causes a change to the neutralizing element. The reconstituting element can be configured to change a property of the neutralizing element, and the property can be selected from the group consisting of: physical; mechanical; chemical; biological; and combinations thereof.

In some embodiments, the reconstituting element comprises water.

In some embodiments, the neutralizing element and the reconstitution element comprise a co-solution.

In some embodiments, the extracellular matrix tissue comprises at least one of sensory nerve tissue, motor nerve tissue, or mixed nerve tissue. The extracellular matrix tissue can further comprise autonomic nerve tissue. The extracellular matrix tissue can further comprise spinal cord nerve tissue. The extracellular matrix tissue can further comprise dorsal root ganglia tissue and/or ventral root ganglia tissue. The extracellular matrix tissue can further comprise sciatic nerve tissue. The sciatic nerve tissue can comprise bilateral sciatic nerve tissue.

In some embodiments, the extracellular matrix tissue comprises tissue harvested from a tissue source selected from the group consisting of: mammals; amphibians; chondrichthyans; reptiles; orcephalopods; and combinations thereof.

In some embodiments, the extracellular matrix exhibits an in vivo degradation rate between 24 hours and six months. The extracellular matrix can exhibit an in vivo degradation rate between two weeks and two months. The extracellular matrix can exhibit an in vivo degradation rate of four weeks.

In some embodiments, the extracellular matrix comprises a fluid comprising a dynamic viscosity between 1 cP and 3000 cP. The extracellular matrix can comprise a fluid comprising a dynamic viscosity between 1 cP and 10 cP. The extracellular matrix can comprise a fluid comprising a dynamic viscosity between 1000 cP and 3000 cP.

In some embodiments, the extracellular matrix is constructed and arranged as a scaffold configured to provide structural support at the treatment site. The scaffold can be configured to provide structural support for a process selected from the group consisting of: cell attachment; cell migration; cell proliferation; cell development; protein secretion; tissue development; and combinations thereof.

In some embodiments, the extracellular matrix is configured to exhibit pharmacological and/or biological properties. The pharmacological and/or biological properties can be configured to promote a process selected from the group consisting of: immunomodulatory action; revascularization; cell chemotaxis; cell development; protein secretion; nerve tissue deposition; and combinations of these.

In some embodiments, the system further comprises one or more vials configured to store at least one of the extracellular matrix, neutralizing element, or reconstituting element. At least one of the one or more vials can include a sterility barrier. The sterility barrier can be configured to prevent the passage of fluid between the vial and an external environment. The sterility barrier can be configured to prevent the passage of contaminants between the vial and an external environment. The sterility barrier can comprise an element selected from the group consisting of: rubber stopper; flip-off cap; tear-off seal; crimp seal; plastic seal; and combinations thereof. The system can further comprise one or more vial stoppers configured to be inserted into an opening of one of the one or more vials. At least one of the one or more vial stoppers can comprise a configuration selected from the group consisting of: two-leg; three-leg; round bottom; igloo; straight plug; and combinations thereof. At least one of the one or more vial stoppers can comprise a surface area configured to prevent the loss of fluid and/or powder within one of the one or more vials. At least one of the one or more vial stoppers can comprise a surface area configured to provide a moisture barrier to the extracellular matrix within at least one of the one or more vials. At least one of the one or more vial stoppers can further include a fluid exchange element configured to allow for the passage of fluid between one of the one or more vials and an external environment. The fluid exchange element can comprise a vent comprising a membrane. The membrane can comprise a selectively permeable membrane. At least one of the one or more vial stoppers can be configured to prevent the passage of fluid between one of the one or more vials and an external environment. At least one of the one or more vial stoppers can be configured to prevent the passage of contaminants between one of the one or more vials and an external environment.

In some embodiments, the system further comprises one or more fluid delivery devices configured to receive and/or expel at least one of the extracellular matrix, neutralizing element, or reconstituting element. The one or more fluid delivery devices can comprise a syringe.

In some embodiments, the system further comprises a lyophilization device configured to dehydrate the extracellular matrix. The lyophilization device can be configured to dehydrate the extracellular matrix to a residual moisture content of between 0.1% and 10%. The lyophilization device can be configured to dehydrate the extracellular matrix to a residual moisture content of less than 4%. The lyophilization device can be configured to dehydrate the extracellular matrix to a residual moisture content of between 2% and 4%. The lyophilization device can be configured to dehydrate the extracellular matrix to a residual moisture content of between 0.2% and 2.5%.

In some embodiments, the system further comprises one or more digestive enzymes configured to alter a property of the extracellular matrix. The digestive enzyme can comprise pepsin comprising an activity level of between 0.5 U/mg and 5000 U/mg. The digestive enzyme can comprise pepsin comprising an activity level of 250 U/mg. The extracellular matrix includes the digestive enzyme, and the digestive enzyme can be configured to alter the gel mechanical properties and/or gelation kinetics of the extracellular matrix. The extracellular matrix including the digestive enzyme can be reconstituted and neutralized with phosphate-buffered saline (PBS) comprising an ionic strength equivalent to 62.5% of that of an isotonic solution. The digestive enzyme can comprise a concentration of 100 U/mL, and the extracellular matrix including the digestive enzyme can comprise a storage modulus of no less than 25 Pa and no more than 40 Pa. The digestive enzyme can comprise a concentration of 250 U/mL, and the extracellular matrix including the digestive enzyme can comprise a storage modulus of no less than 90 Pa and no more than 140 Pa. The digestive enzyme can comprise a concentration of 500 U/mL, and the extracellular matrix including the digestive enzyme can comprise a storage modulus of no less than 85 Pa and no more than 125 Pa. The digestive enzyme can comprise a concentration of 1000 U/mL, and the extracellular matrix including the digestive enzyme can comprise a storage modulus of no less than 100 Pa and no more than 155 Pa. The extracellular matrix including the digestive enzyme can be reconstituted and neutralized with phosphate-buffered saline (PBS) comprising an ionic strength equivalent to 50% of that of an isotonic solution. The digestive enzyme can comprise a concentration of 100 U/mL, and the extracellular matrix including the digestive enzyme can comprise a storage modulus of no less than 45 Pa and no more than 75 Pa. The digestive enzyme can comprise a concentration of 250 U/mL, and the extracellular matrix including the digestive enzyme can comprise a storage modulus of no less than 155 Pa and no more than 220 Pa. The digestive enzyme can comprise a concentration of 500 U/mL, and the extracellular matrix including the digestive enzyme can comprise a storage modulus of no less than 120 Pa and no more than 190 Pa. The digestive enzyme can comprise a concentration of 1000 U/mL, and the extracellular matrix including the digestive enzyme can comprise a storage modulus of no less than 120 Pa and no more than 180 Pa. The extracellular matrix includes the digestive enzyme, and the digestive enzyme can comprise a concentration configured to alter the shelf life of the extracellular matrix at a storage temperature of between 2° C. and 25° C. The digestive enzyme can comprise a concentration of 100 U/mL, and the extracellular matrix including the digestive enzyme can comprise a shelf life of more than three months at storage temperature of between 20° C. and 25° C. The digestive enzyme can comprise a concentration of 1000 U/mL, and the extracellular matrix including the digestive enzyme can comprise a shelf life of no more than one month at storage temperature of between 20° C. and 25° C.

In some embodiments, the system further comprises one or more excipients configured to enhance a property of the extracellular matrix. The one or more excipients can be configured to enhance a property of the extracellular matrix selected from the group consisting of: long-term stabilization; bulking; radioprotection; heat protection; cryoprotection; lyoprotection; solubility; and combinations thereof. The one or more excipients can be selected from the group consisting of: sucrose; ascorbic acid; sodium ascorbate; sodium azide; Vitamin E; ethylenediaminetetraacetic acid (EDTA); mannitol; glycerol; and combinations thereof. The one or more excipients can be configured to increase a relative solubility of the extracellular matrix. The one or more excipients can be configured to increase a relative gelation of the extracellular matrix.

In some embodiments, the system further comprises one or more radioprotective agents configured to reduce free radical damage of the extracellular matrix when exposed to ionizing radiation. The one or more radioprotective agents can be selected from the group consisting of: Vitamin E and/or Vitamin E derivatives comprising a concentration of between 0.01 mg/mL and 50 mg/mL; ascorbic acid comprising a concentration of between 0.01 mg/mL and 50 mg/mL; glycerol comprising a concentration of between 0.1 mg/mL and 10 mg/mL; riboflavin comprising a concentration of between 0.05 mg/mL and 10 mg/mL; polyvinylpyrrolidone (PVP) comprising a concentration of between 0.05 mg/mL and 10 mg/mL; sodium ascorbate comprising a concentration of between 0.005 mg/mL and 40 mg/mL; sodium azide comprising a concentration of between 0.03 mg/mL and 15 mg/mL; hydroquinone comprising a concentration of between 0.2 mg/mL and 35 mg/mL; and combinations thereof.

According to another aspect of the present inventive concepts, a method for producing an extracellular matrix comprises: harvesting tissue from a tissue source, processing the tissue to produce a raw material; decellularizing the raw material to produce an extracellular matrix; lyophilizing the extracellular matrix; mechanically disrupting the lyophilized extracellular matrix; and digesting the disrupted extracellular matrix. The digested extracellular matrix can be configured to provide a therapeutic benefit to the patient.

In some embodiments, the method further comprises aliquoting the digested extracellular matrix between one, two, or more containers. Each container can receive between 0.25 mL and 5 mL of the digested extracellular matrix. The container can comprise a vial. A vial stopper can be inserted into an opening of the vial. The vial stopper can further include a fluid exchange element configured to allow for the passage of fluid between the vial and an external environment. The vial stopper can be configured to prevent the passage of fluid between the vial and an external environment. The container can comprise a syringe. The container can be sterilized prior to receiving the digested extracellular matrix. The method can further comprise lyophilizing the containers comprising the digested extracellular matrix. The method can further comprise packaging the containers comprising the digested extracellular matrix. The package containers comprising the digested extracellular matrix can be stored at a temperature of between 2° C. and 8° C. The method can further comprise sterilizing the packaged containers comprising the digested extracellular matrix, and the sterilization can comprise exposing the containers to gamma irradiation. The packaged containers comprising the digested extracellular matrix can be exposed to gamma irradiation in one or more doses of between 8 kGy and 25 kGy. The method can further comprise sterilizing the packaged containers comprising the digested extracellular matrix, and the sterilization can comprise exposing the containers to electron-beam irradiation. The packaged containers comprising the digested extracellular matrix can be exposed to beta radiation in one or more doses of between 8 kGy and 25 kGy. The method can further comprise sterilizing the packaged containers comprising the digested extracellular matrix, and the sterilization can comprise exposing the containers to a supercritical carbon dioxide gas. The method can further comprise sterilizing the packaged containers comprising the digested extracellular matrix, and the sterilization can comprise exposing the containers to an ethylene oxide gas. The method can further comprise sterilizing the packaged containers comprising the digested extracellular matrix, and the sterilization can comprise exposing the containers to a vaporized peracetic acid. The method can further comprise sterilizing the packaged containers comprising the digested extracellular matrix, and the sterilization can comprise exposing the containers to a nitrogen dioxide gas.

In some embodiments, the method is performed within an environment suitable for aseptic processing. The method can be performed in a sterile work area configured to prevent contamination from microorganisms.

In some embodiments, the tissue is harvested from two, three, or more tissue sources. The tissue can be harvested from the two, three, or more tissue sources can be pooled together to provide a larger quantity of tissue.

In some embodiments, the harvested tissue comprises at least one of sensory, motor, or mixed nerve tissue.

In some embodiments, processing the tissue comprises removing connective and/or accessory tissue to produce the raw material. The raw material can be further processed to remove additional connective and/or accessory tissue. The raw material can be further processed at a temperature of between 2° C. and 25° C. The raw material can comprise a final mass:initial mass ratio of at least 1:2, and the removed connective and/accessory tissue can comprise less than 50% of the raw material initial mass.

In some embodiments, the raw material is at least partially immersed in a buffer solution for short-term storage. The short-term storage can comprise a duration of less than six hours. The buffer solution can comprise phosphate-buffered saline (PBS).

In some embodiments, the raw material is stored at a temperature of between 2° C. and 8° C.

In some embodiments, the raw material is rapidly frozen in a buffer solution for long term storage and/or transportation. The long-term storage and/or transportation can comprise a duration of more than six hours. The raw material can be rapid frozen via a cooling agent selected from the group consisting of: dry ice; dry ice with ethanol; dry ice with acetone; liquid nitrogen; wet ice; frozen ice packs; cold packs; and combinations thereof.

In some embodiments, the raw material is stored and/or transported at a temperature of −80° C. The raw material can be stored and/or transported at −80° C. for a maximum of six months.

In some embodiments, the raw material is cut into smaller segments. The raw material can be cut into segments between 0.5 cm and 2 cm.

In some embodiments, the raw material is transferred into one, two, or more vessels. The raw material can be transferred at a temperature of between 2° C. and 25° C. Each vessel can receive no more than 25 g of the raw material.

In some embodiments, the raw material is washed with purified water. The raw material can be washed with purified water at a temperature of between 2° C. and 8° C. The raw material can be washed with purified water at least two times. The raw material can be washed with purified water at least three times. The raw material can be washed with purified water at least four times. The raw material and purified water can comprise a ratio of between 1:20 and 1:50. The raw material can be washed with purified water overnight.

In some embodiments, decellularizing the raw material comprises washing the raw material with a dissociation solution. The dissociation solution can comprise a co-solution comprising trypsin and ethylenediaminetetraacetic acid (EDTA). Washing the raw material with the dissociation solution forms a lipid layer on the surface of the dissociation solution, and the lipid layer can be removed via an instrument. The instrument can be selected from the group consisting of: pipette; forceps; scalpel; scraper; blade; and combinations thereof.

In some embodiments, lyophilizing the extracellular matrix comprises dividing and transferring the extracellular matrix into one, two, or more lyophilization receptacles. The extracellular matrix can be transferred into the lyophilization receptacles via a depyrogenated instrument. The lyophilization receptacles comprising the extracellular matrix can be inserted into a lyophilization pouch. The lyophilization receptacles comprising the extracellular matrix can be loaded into a lyophilization device configured to perform a lyophilization process. The lyophilization process can comprise a duration of between 12 and 66 hours. The lyophilization process can comprise a duration of between 18 and 24 hours. The lyophilization process can comprise a duration of approximately 24 hours. The lyophilization device can be configured to freeze the lyophilization receptacles comprising the extracellular matrix at a temperature of −40° C. for no less than four hours. The lyophilization device can be configured to apply vacuum source to the lyophilization receptacles comprising the extracellular matrix. The vacuum source can comprise 150 micrometers of Hg. The lyophilization device can be configured to dry the lyophilization receptacles comprising the extracellular matrix at a temperature of between −8° C. and 0° C.

In some embodiments, mechanically disrupting the lyophilized extracellular matrix comprises dividing and transferring the extracellular matrix into one, two, or more tubes. Each tube can receive 10±1 g of the lyophilized extracellular matrix. The tubes can be transferred to a batch mill configured to grind the lyophilized extracellular matrix within the tubes. The batch mill can comprise a grinding speed of between 5000 rpm and 50,000 rpm. The batch mill can be configured to grind the lyophilized extracellular matrix in time intervals. The time intervals can comprise a duration of between five seconds and 60 seconds. The time intervals can comprise between one and five time intervals. The lyophilized extracellular matrix can be ground via the batch mill to achieve a desired morphology. The desired morphology can comprise a fiber-like morphology. The desired morphology can be further refined via size exclusion.

In some embodiments, digesting the disrupted extracellular matrix comprises dividing and transferring the extracellular matrix into one, two, or more bottles. Each bottle can receive between 1 g and 20 g of the disrupted extracellular matrix. Each bottle can receive 8.4 g of the disrupted extracellular matrix. The bottle can be sterilized prior to receiving the disrupted extracellular matrix. A digestion solution can be added to the bottles including the disrupted extracellular matrix. The digestion solution can comprise an acid solution and a digestive enzyme. The acid solution can comprise a 0.01 N hydrochloric acid (HCl) solution. The digestive enzyme can comprise pepsin. Each bottle can receive between 250 mL and 1000 mL of the digestion solution. The collective volume of the disrupted extracellular matrix and the digestion solution can comprise greater than or equal to 70% of the total volume of the bottle. The final concentration of the disrupted extracellular matrix in the digestion solution can comprise between 0.5 mg/mL and 100 mg/mL. The bottles comprising the disrupted extracellular matrix and the digestive solution can be stored at a temperature of between 2° C. and 37° C. for a duration of at least 12 hours, and a digested extracellular matrix can be produced. The bottles comprising the disrupted extracellular matrix and the digestive solution can be stored at a temperature of between 15° C. and 30° C. The bottles comprising the disrupted extracellular matrix and the digestive solution can be stored at a temperature of between 18° C. and 23° C. The bottles comprising the disrupted extracellular matrix and the digestive solution can be stored for a duration between 46 and 50 hours. A mixing device can be lowered into each bottle, and the mixing device can be configured to stir the disrupted extracellular matrix and the digestive solution at a speed of between 100 rpm and 5000 rpm. The mixing device can be configured to stir the disrupted extracellular matrix and the digestive solution at a speed of 1400 rpm for a duration of at least 12 hours. An initial pH of the digested extracellular matrix can be adjusted to comprise a target pH. The target pH can be configured to alter the shelf life of the digested extracellular matrix. The target pH can be configured to alter the solubility of the digested extracellular matrix. The target pH can comprise a pH greater than 7.4. An initial volume of the digested extracellular matrix can be adjusted to comprise a target volume. The target volume can comprise 900 mL. One, two, or more excipients can be added to the digested extracellular matrix. One, two, or more radioprotectants can be added to the digested extracellular matrix.

According to another aspect of the present inventive concepts, a method for treating a patient comprising: deploying an extracellular matrix at a deposit site in the patient. The extracellular matrix can be configured to provide a therapeutic benefit at a treatment site.

In some embodiments, the extracellular matrix is configured to provide a therapeutic benefit at two or more treatment sites.

In some embodiments, the deposit site is proximate the treatment site.

In some embodiments, the extracellular matrix is deployed at two or more deposit sites.

In some embodiments, the extracellular matrix is deployed to extend to a location beyond the deposit site. The extracellular matrix can be deployed to extend to one, two, or more locations beyond the deposit site. The extracellular matrix can be deployed to extend longitudinally beyond the deposit site. The extracellular matrix can be deployed to extend proximally from the deposit site. The extracellular matrix can extend between 2 mm and 20 mm proximally from the deposit site. The extracellular matrix can be deployed to extend distally from the deposit site. The extracellular matrix can extend between 2 mm and 20 mm distally from the deposit site.

In some embodiments, the deposit site is proximate a nerve. The extracellular matrix can be deployed at multiple deposit sites about the circumference of the nerve. The multiple deposit sites can comprise a uniform spacing about the circumference of the nerve. The extracellular matrix can be deployed at three deposit sites about the circumference of the nerve, and the second deposit site can be 120° relative to the first deposit site, and the third deposit site can be 120° relative to the second deposit site, and the third deposit site can be 240° relative to the first deposit site. The multiple deposit sites can comprise a non-uniform spacing about the circumference of the nerve. The extracellular matrix can be further deployed to extend to a location beyond the multiple deposit sites, and the deployment of the extracellular matrix can comprise a matrix along an external surface of the nerve.

In some embodiments, the deposit site comprises a location within the peripheral nervous system. The deposit site can comprise a location that can be not within the brain and spinal cord.

In some embodiments, the deposit site comprises a location within and/or proximate an uninjured nerve.

In some embodiments, the deposit site comprises a location within and/or proximate a diseased nerve.

In some embodiments, the deposit site comprises a location within and/or proximate a nerve injury.

In some embodiments, the deposit site comprises a location within and/or proximate a partial nerve transection.

In some embodiments, the deposit site comprises a location within and/or proximate a full nerve transection.

In some embodiments, the deposit site comprises a location within and/or proximate a nerve transfer.

In some embodiments, the deposit site comprises a location within and/or proximate a nerve crush injury.

In some embodiments, the deposit site comprises a location within and/or proximate a nerve stretch injury.

In some embodiments, the deposit site comprises a location within and/or proximate a compression nerve injury.

In some embodiments, the extracellular matrix is deployed contemporaneously with a structural element. The extracellular matrix can be deployed contemporaneously with sutures. The extracellular matrix can be deployed contemporaneously with a conduit. The extracellular matrix can be deployed contemporaneously with a wrap. The extracellular matrix can be deployed contemporaneously with glue.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of a system for producing and deploying a medical device comprising an extracellular matrix, consistent with the present inventive concepts.

FIG. 2 illustrates a perspective view of a medical device comprising a conduit, consistent with the present inventive concepts.

FIG. 3 illustrates a method for producing an extracellular matrix from tissue, consistent with the present inventive concepts.

FIG. 4 illustrates a method for harvesting and/or preparing tissue for further manipulation, consistent with the present inventive concepts.

FIG. 5 illustrates a method for decellularizing tissue to produce an extracellular matrix, consistent with the present inventive concepts.

FIG. 6 illustrates a method for lyophilizing an extracellular matrix, consistent with the present inventive concepts.

FIG. 7 illustrates a method for mechanically disrupting an extracellular matrix, consistent with the present inventive concepts.

FIG. 8 illustrates a method for digesting an extracellular matrix, consistent with the present inventive concepts.

FIG. 9 illustrates a method for aliquoting an extracellular matrix between one, two, or more containers, consistent with the present inventive concepts.

FIG. 10 illustrates a method for lyophilizing a container comprising an extracellular matrix, consistent with the present inventive concepts.

FIG. 11 illustrates a method for packaging and storing a container comprising an extracellular matrix, consistent with the present inventive concepts.

FIG. 12 illustrates another method for digesting an extracellular matrix, consistent with the present inventive concepts.

FIG. 13 illustrates another method for aliquoting an extracellular matrix between one, two, or more containers, consistent with the present inventive concepts.

FIG. 14 illustrates another method for lyophilizing a container comprising an extracellular matrix, consistent with the present inventive concepts.

FIG. 15 illustrates another method for packaging and storing a container comprising an extracellular matrix, consistent with the present inventive concepts.

FIG. 16 illustrates a method for an irradiation based sterilization of a container comprising an extracellular matrix, consistent with the present inventive concepts.

FIG. 17 illustrates another method for lyophilizing a container comprising an extracellular matrix, consistent with the present inventive concepts.

FIG. 18 illustrates another method for packaging and storing a container comprising an extracellular matrix, consistent with the present inventive concepts.

FIG. 19 illustrates a method for a gas based sterilization of a container comprising an extracellular matrix, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

As used herein, the term “functional element” is to be taken to include one or more elements constructed and arranged to perform a function. A functional element can comprise a sensor and/or a transducer. In some embodiments, a functional element is configured to generate and/or deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of two or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

As used herein, the term “fluid” can refer to a liquid, gas, gel, or any flowable material, such as a material which can be propelled through a lumen and/or opening.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are improved nerve injury treatment systems, devices and methods.

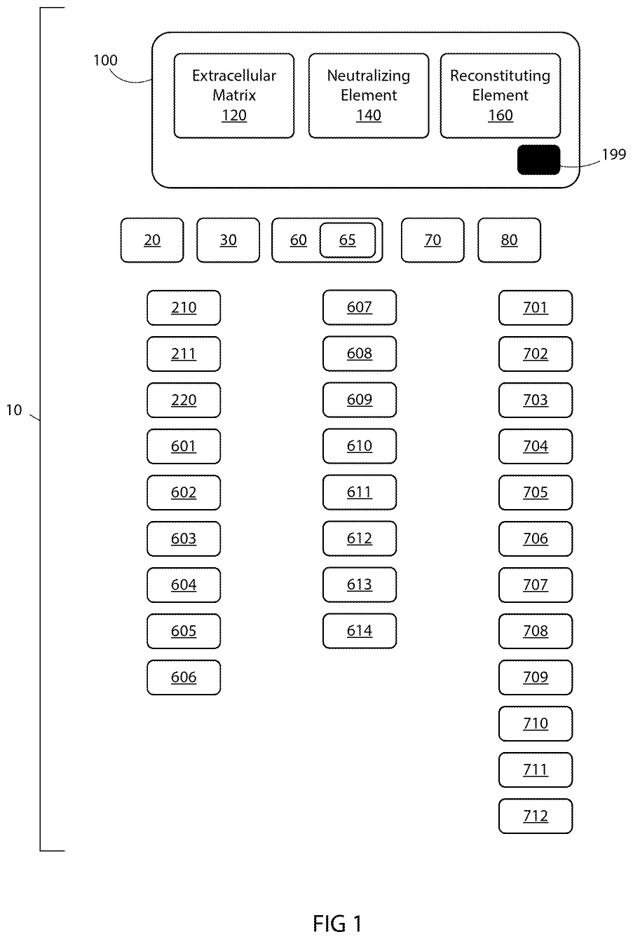

Referring now to FIG. 1, a schematic view of a system for producing and deploying a medical device comprising an extracellular matrix is illustrated, consistent with the present inventive concepts. System 10 comprises medical device 100 shown, as well as various components used to manufacture, package, sterilize, and/or deploy device 100. Device 100 is configured to be deployed (e.g. injected, inserted, implanted, and the like) at one, two, or more “deposit sites”, such as to provide a therapeutic benefit at one, two, or more “treatment sites”. Each treatment site can comprise a location that is proximate to and/or remote from the associated deposit site. In some embodiments, a treatment site comprises a location that is relatively the same location as the associated deposit site. Device 100 can be deployed at the deposit site to promote, and/or otherwise support, tissue growth of a patient (e.g. support tissue growth and/or regeneration at locations proximate and/or remote from the deposit site). In some embodiments, device 100 is remodeled over time into native tissue of the patient. As used herein, the deposit site can comprise one, two, or more locations on and/or within the patient.

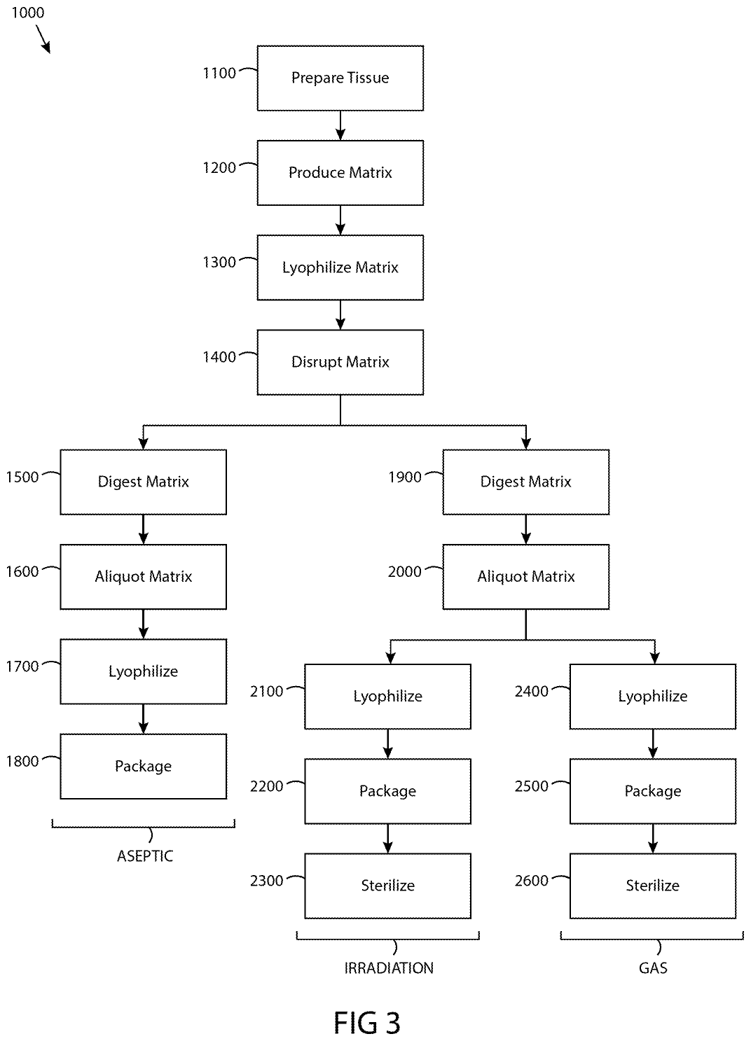

Device 100 comprises a decellularized extracellular matrix, ECM 120 shown. ECM 120 can comprise structural and non-structural biomolecules, including, but not limited to, collagens, elastins, laminins, glycosaminoglycans, proteoglycans, antimicrobials, chemoattractants, cytokines, and growth factors. ECM 120 can be configured to promote and/or sustain the growth of tissue and/or associated tissue properties (e.g. structural proteins, growth factors, etc.) proximate to and/or remote from the deposit site. ECM 120 can be derived, or otherwise produced, from one, two, or more raw material 65 as described herein. In some embodiments, ECM 120 is derived from raw material 65 according to Methods 1000-2600 as described herein in reference to FIGS. 3-19, respectively. ECM 120 can comprise a concentration of native protein between 5 mg/mL and 50 mg/mL, such as a concentration between 10 mg/mL and 30 mg/mL, such as a concentration of approximately 20 mg/mL. The protein concentration can be configured to improve a parameter of ECM 120, such as to improve solubility, reconstitution, mechanical modulus, in vivo remodeling, and/or durability.

Device 100 can further comprise a neutralizing element 140 and/or a reconstituting element 160, each configured to interact (e.g. physically, chemically interact) with ECM 120. In some embodiments, neutralizing element 140 and/or reconstituting element 160 interact with ECM 120 to cause a physical and/or chemical change to ECM 120 and/or other component of system 10. Neutralizing element 140 can be configured to counteract, or otherwise offset, a property (e.g. physical, mechanical, chemical, and biological property) of ECM 120, reconstituting element 160, and/or other component of system 10. In some embodiments, neutralizing element 140 comprises a buffer of a base configured to neutralize an acid solution. Neutralizing element 140 can comprise an element selected from the group consisting of: water; phosphate-buffered saline (PBS); sodium hydroxide (NaOH); and combinations of these. In some embodiments, ECM 120 comprises a fluid and neutralizing element 140 comprises a concentration of PBS that is configured to modify (e.g. increase, decrease) the mechanical strength of ECM 120, modify (e.g. increase, decrease) a gelation time of ECM 120, and/or modify (e.g. increase, decrease) a gelation temperature of ECM 120. In some embodiments, neutralizing element 140 comprises a solution comprising 0.2 M NaOH and 0.5-1.0×PBS in water. Reconstituting element 160 can be configured to modify, or otherwise change, a property (e.g. physical, chemical, mechanical, and/or biological property) of ECM 120, neutralizing element 140, and/or other component of system 10. Reconstituting element 160 can comprise water. In some embodiments, neutralizing element 140 and reconstituting element 160 are combined to comprise a single solution, such as a co-solution of neutralizing element 140 and reconstituting element 160.

Raw material 65 can comprise sensory, motor, and/or mixed nerve tissue. In some embodiments, raw material 65 comprises autonomic nerve tissue. In some embodiments, raw material 65 comprises spinal cord nerve tissue. In some embodiments, raw material 65 comprises dorsal and/or ventral root ganglia. In some embodiments, raw material 65 comprises sciatic nerve tissue, such as bilateral sciatic nerves. Tissue harvested from multiple (e.g. two, three, or more) nerve types can be pooled to provide a larger quantity and/or heterogenous raw material 65. Raw material 65 can comprise tissue harvested from a tissue source 60 selected from the group consisting of: mammals, such as pig, human, cow, horse, and the like; amphibians, such as salamander and the like; chondrichthyans, such as shark and the like; reptiles, such as and the like; orcephalopods, such as squid and the like; and combinations of these.

Device 100, comprising ECM 120, can comprise a configuration selected from the group consisting of: a fluid and/or semi-fluid (either or both, “fluid” herein), such as a hydrogel, cream, ointment, or the like; a spongy material; a compressed material, such as a film; a solid material, such as a wrap, conduit, graft, suture, or the like; an aerosolized material, such as a spray; a flowable particulate, such as a micronized and flowable particulate; a fibrous material; and combinations of these. In some embodiments, device 100 is configured to deliver one, two, or more therapeutic agents (e.g. agent 70 described herein) to the patient (e.g. pharmaceutical drugs, stem cell therapies, etc.), such was when device 100 further comprises a plurality of microspheres comprising a therapeutic agent.

Device 100 can comprise a mechanical strength that is increased via at least one of chemical cross-linking or physical cross-linking.

Device 100 can comprise a degradation rate in vivo of between 24 hours and six months, such as a degradation rate in vivo of between two weeks and two months, such as a degradation rate in vivo of approximately four weeks.

In some embodiments, device 100 comprises a fluid comprising a dynamic viscosity between 20 cP and 200 cP. Device 100 can comprise a lower viscosity configured for injectable applications, such as a viscosity of between 1 cP and 10 cP. Device 100 can comprise a greater viscosity for topical applications, such as a viscosity of between 1000 cP and 3000 cP.

In some embodiments, device 100 comprises a semi-fluid and/or solid that is molded, or otherwise manipulated, into a geometric shape prior to, during, and/or after deployment at the deposit site.

In some embodiments, device 100 is constructed and arranged as a coating configured to at least partially cover one, two, or more surfaces of the deposit site. Device 100 can be configured to coat a surface of the deposit site via an atomization process, such as an atomization process performed using tool 80. Device 100 can be configured to coat a surface of the deposit site via a brushing process, such as a brushing process performed using tool 80. Device 100 can be configured to coat a surface of the deposit site via a dipping process, such as a dipping process performed using tool 80.

In some embodiments, device 100 is constructed and arranged as a scaffold configured to provide structural support for cell attachment, migration, proliferation, development, protein secretion, and/or tissue development at a treatment site.

Device 100 can be incorporated into (e.g. embedded in, combined with, used in conjunction with, and the like) an existing medical device and/or material. In some embodiments, device 100 is incorporated into a patch and/or film. In some embodiments, device 100 is incorporated into a nerve guide. In some embodiments, device 100 is incorporated into a nerve conduit.

Device 100 can be delivered, injected, implanted, and/or otherwise deployed (“deployed” herein) proximate a treatment site. Device 100 can be deployed into, onto, and/or at the deposit site, such as a focal area of a treatment site.

Device 100 can be deployed to extend to, or otherwise cover, one, two, or more locations beyond the deposit site (e.g. into locations of the treatment site or other locations). Device 100 can be deployed to extend longitudinally beyond the deposit site. In some embodiments, device 100 extends proximally from the deposit site, such as between 2 mm and 20 mm proximally from the deposit site, such as between 2 mm and 5 mm, such as between 5 mm and 10 mm, such as between 10 mm and 20 mm. In some embodiments, device 100 extends distally from the deposit site, such as between 2 mm and 20 mm distally from the deposit site, such as between 2 mm and 5 mm, such as between 5 mm and 10 mm, such as between 10 mm and 20 mm.

Device 100 can be deployed at multiple (e.g. two, three, or more) deposit sites positioned about the circumference of a nerve. Two or more deposit sites can comprise a uniform spacing about the circumference of the nerve. For example, device 100 can be deployed at a first deposit site representing 0°, at a second deposit site is 120° relative to the first deposit site, and a third deposit site that is 240° relative to the first deposit site (and 120° relative to the second deposit site). The two or more deposit sites can comprise a non-uniform spacing about the circumference of the nerve.

Device 100 can be deployed at one, two, or more deposit sites about the circumference of a nerve and can be further deployed at one, two, or more locations beyond the deposit sites, as described herein. In some embodiments, deployment of device 100 at the deposit sites and locations beyond the deposit sites comprise a matrix of device 100 along the external surface of the nerve. In some embodiments, the deposit site comprises a location (e.g. one, two, three, or more locations) within the central nervous system, such as a site located within the brain and/or spinal cord.

In some embodiments, the deposit site comprises a location within the peripheral nervous system, such as a site that is not within the brain and spinal cord, including any location along the peripheral nervous system spanning from the dorsal and/or ventral root ganglia to motor, sensory, autonomic endings (e.g. end-muscle plates, Pacinian corpuscles, Ruffini endings, etc.).

In some embodiments, the deposit site comprises a location within and/or proximate an uninjured nerve. In some embodiments, the deposit site comprises a location within and/or proximate a diseased nerve. In some embodiments, the deposit site comprises a location within and/or proximate a nerve injury, such as an intra-nerve and/or peri-nerve injury location. In some embodiments, the deposit site comprises a location proximate a partial or full nerve transection. For example, device 100 can be deployed to provide an interface between two or more nerve stumps. In some embodiments, the deposit site comprises a location proximate a nerve transfer, such as an end-to-end transfer, side-to-side transfer, end-to-side transfer, supercharge end-to-side transfer. In some embodiments, the deposit site comprises a location proximate a nerve crush injury, such as an acute crush injury. In some embodiments, the deposit site comprises a location proximate a nerve stretch injury, such as an acute stretch injury. In some embodiments, the deposit site comprises a location proximate a compression nerve injury, such as chronic compression with or without surgical release.

In some embodiments, device 100 is deployed into a deposit site comprising oral tissue (e.g. oral mucosa, teeth, tooth pulp, cranial nerve, tongue). In some embodiments, device 100 is deployed into the tooth root following a root canal or pulpectomy procedure. In some embodiments, device 100 is deployed into and/or around the cranial nerves. In some embodiments, device 100 is deployed into the oral mucosa or tongue.

Device 100 can be deployed contemporaneously (e.g. concurrently) with one, two, or more additional treatments provided to the patient (e.g. one or more treatments deployed at the deposit site, the treatment site, and/or another patient location). In some embodiments, device 100 is deployed contemporaneously with an electrical stimulation. In some embodiments, device 100 is deployed contemporaneously with a pharmacological treatment. In some embodiments, device 100 is deployed contemporaneously with a cellular treatment. In some embodiments, device 100 is deployed contemporaneously with a structural element (e.g. sutures, conduit, wrap, glue).

Device 100 can comprise one or more functional elements, functional element 199 shown. Functional element 199 can comprise a sensor and/or a transducer. In some embodiments, functional element 199 comprises a biofeedback element. For example, device 100 can further comprise a biofeedback mechanism (e.g. functional element 199) configured to provide an indication of a biological event at the deposit site.

In some embodiments, device 100 further comprises one or more pharmacological or other agents, agent 70 shown. Agent 70 can comprise a chemoattractant configured to attract motile cells to the deposit site, such as a motile cell selected from the group consisting of: Schwann cells; macrophages; endothelial cells; progenitor cells; and combinations of these. Agent 70 can comprise an agent configured to promote the production of angiogenic factors at the deposit site, such as an angiogenic factor selected from the group consisting of: angiogenic; growth factors, such as fibroblast growth factors, transforming growth factors, and the like; lipids; and combinations of these. Agent 70 can comprise an agent configured to promote cell migration, development, and/or maturation at the deposit site, such as a nerve growth factor.

In some embodiments, device 100 is configured to exhibit pharmacological and/or biological properties configured to support the local microenvironment at the deposit site, such as to promote immunomodulatory action, revascularization, cell chemotaxis, cell development, protein secretion, nerve tissue deposition, and/or combinations of these.

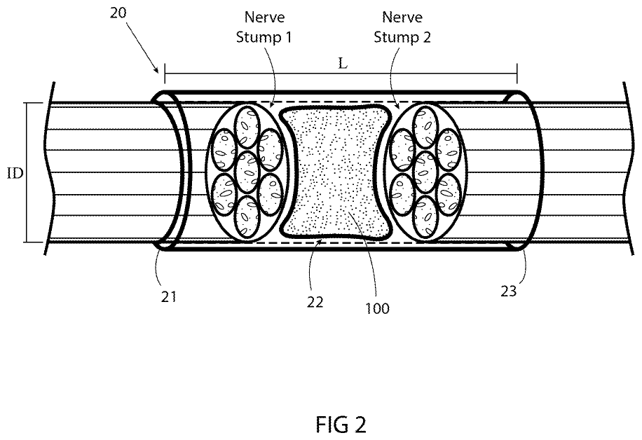

System 10 can further comprise one or more implants, implant 20 shown. Implant 20 can comprise a conduit as described herein in reference to FIG. 2.

System 10 can further comprise one or more imaging devices, device 30 shown, which can be configured to visualize an object (e.g. device 100). Device 30 can comprise an imaging device selected from the group consisting of: microscope; loupe; device that provides virtual reality visualization; device that provides stereo visualization; device that provides infrared near-infrared visualization; device that provides thermal imaging; medical imaging device, such as an X-ray, a fluoroscope, an Mill, a CT scanner, an ultrasound, an endoscope; device that images using UV light; device that images using polarized light; device that images using fluorescent light; and combinations of these.

System 10 can further comprise one or more tools, tool 80 shown, which can be configured to coat a surface (e.g. a surface of a deposit site), such as an atomization tool, a brush or brushing tool, and/or a dipping tool, as described herein. Tool 80 can comprise a tattoo machine configured to deliver ECM 120 at a defined depth of a surface. Tool 80 can comprise a jet injector configured to deliver ECM 120 via high pressure of at a defined depth of a surface. Tool 80 can comprise a bobbin including a coiled strand or ribbon embedded with ECM 120 configured to wind and/or canvas around a surface. Tool 80 can comprise an adhesive or adhesive strip configured to affix ECM 120 to a surface.

System 10 can further comprise one or more vials, vial 210 shown, which can be configured to store one, two, or more fluids, powders, cakes, microspheres, and/or capsules. Vial 210 can be configured to store a volume between 0.5 mL and 50 mL, such as a volume between 2 mL and 5 mL. Vial 210 can comprise a material selected from the group consisting of: glass, such as Type 1 borosilicate; plastic, such as polypropylene, polyethylene; polyolefins, cyclic olefin copolymers, metal, such as stainless steel, aluminum; and combinations of these.

Vial 210 can further include a sterility barrier 211 configured to prevent or otherwise reduce the passage of fluid between vial 210 and an external environment. In some embodiments, sterility barrier 211 is configured to prevent or otherwise reduce the passage of contaminants (e.g. bacteria, virus, dust particles, etc.) between vial 210 and an external environment. Sterility barrier 211 can comprise an element selected from the group consisting of: a rubber stopper; a flip-off cap, such as a plastic cap; a tear-off seal, such as an aluminum seal; a crimp seal, such as a plastic seal; and combinations of these.

System 10 can further comprise one or more vial stoppers, stopper 215 shown, which can be configured to be inserted into an opening of vial 210. Stopper 215 can comprise a configuration selected from the group consisting of: multiple leg, such as two-leg, three-leg, and the like; round bottom; igloo; straight plug; and combinations of these. Stopper 215 can comprise a surface area configured to prevent, or otherwise reduce, the loss of a fluid and/or powder within vial 210. Stopper 215 can comprise a surface area configured to provide a moisture barrier to the fluid and/or powder within vial 210. Stopper 215 can further include a fluid exchange element configured to allow for the passage of fluid between vial 210 and an external environment. In some embodiments, the fluid exchange element comprises a vent that can further comprise a membrane (e.g. a selectively permeable membrane, such as membrane permeable to gas but impermeable to microorganisms). Stopper 215 can be configured to prevent or otherwise reduce the passage of fluid between vial 210 and an external environment, such as when stopper 215 does not include a fluid exchange element. Stopper 215 can be configured to prevent or otherwise reduce the passage of contaminants (e.g. germs, dust particles, etc.) between vial 210 and an external environment.

System 10 can further comprise one or more fluid delivery devices, syringe 220 shown, which can be configured to draw in, or otherwise receive, and/or expel a fluid. Syringe 220 can comprise a barrel 202 configured to receive a plunger 212. Barrel 202 can comprise a distal end comprising a Luer Lock 204 and a proximal end comprising a barrel flange 206. Plunger 212 can comprise a distal end comprising a seal 214 and a proximal end comprising a plunger flange 216. In some embodiments, plunger 212 comprises a removable seal 214, such as that seal 214 can be detached from plunger 212 and positioned within barrel 202. Syringe 220 can comprise a material selected from the group consisting of: plastic, such as polyolefins, cyclic olefin copolymer; glass; and combinations of these. Syringe 220 can include a pre-attached (e.g. pre-inserted) plunger 212. Alternatively, syringe 220 may not include a pre-attached plunger 212, such that a separate plunger 212 is provided for subsequent attachment to syringe 220. In some embodiments, syringe 220 comprises a tuberculin syringe that can receive up to 1 mL of fluid.

Syringe 220 can be configured to receive, maintain (e.g. surround), and/or deploy device 100 to the deposit site, such as device 100 comprising a fluid. An operator can manipulate syringe 220 to control at least one of the following: angle of deployment; depth of deployment; volume of deployment; flow rate of deployment; positioning of deployment; pattern of deployment (e.g. beads, lines, helix, matrix); or combinations of these.

System 10 can further comprise one or more environmental chambers, chamber 601 shown. Chamber 601 can comprise a temperature-controlled environmental chamber configured to chill and/or freeze an object (e.g. raw material 65) through non-cyclic and/or cyclic refrigeration. In some embodiments, chamber 601 comprises frozen ice or synthetic ice packs within an insulated container. In some embodiments, chamber 601 comprises a refrigerator or deli case with or without an incorporated shaker system.

System 10 can further comprise one or more vessels, vessel 602 shown, which can be configured to store an object (e.g. raw material 65). Vessel 602 can comprise a vented container configured to comprise one or more openings to allow for the passage of air, gas, and/or liquid through vessel 602. In some embodiments, vessel 602 comprises one, two, or more pre-sterilized, semi-closed, and/or single-use systems. In some embodiments, vessel 602 is configured to store a tissue sample during tissue processing, embedding, and/or sectioning.

System 10 can further comprise one or more mixing devices, device 603 shown, which can be configured to stir, mix, and/or otherwise agitate a fluid disposed within a component of mixing device 603. In some embodiments, mixing device 603 is further configured to warm and/or maintain the temperature of the fluid. In some embodiments, mixing device 603 comprises a pre-sterilized, semi-closed, and/or single-use system. Mixing device 603 can be configured to agitate a fluid at a speed between approximately 50 rpm and 1,000 rpm, such as approximately 120 rpm. In some embodiments, mixing device 603 includes an impeller configured to rotate, thereby agitating a fluid disposed within mixing device 603. In some embodiments, mixing device 603 comprises an ultrasonic mixing device configured to produce mechanical shock waves.

System 10 can further comprise one or more heating devices 604, device 604 shown. Heating device 604 can be configured to warm and/or maintain the temperature of an object (e.g. raw material 65). In some embodiments, heating device 604 is further configured to stir, mix, and/or otherwise agitate the object. Heating device 604 can comprise a hotplate comprising electric heating elements. In some embodiments, heating device 604 comprises a stirring hotplate comprising a rotating magnetic field configured to rotate a corresponding magnetic bar that is positioned in fluid proximate a surface of heating device 604. In some embodiments, heating device 604 comprises an incubator with or without an incorporated shaking and/or mixing system.

System 10 can further comprise one or more laboratory instruments, instrument 605 shown, such as an instrument selected from the group consisting of: pipette, such as a serological pipette, a positive displacement pipette; forceps, such as serrated tip forceps, single tooth forceps; scalpel, such as a stainless-steel scalpel; scraper, such as a stainless-steel scraper; blade, such as a stainless-steel blade; a cutting surface, such as a polymeric cutting board; band, such as silicone band; funnel; temperature probe; a measuring device, such as a ruler or caliper; and combinations of these.

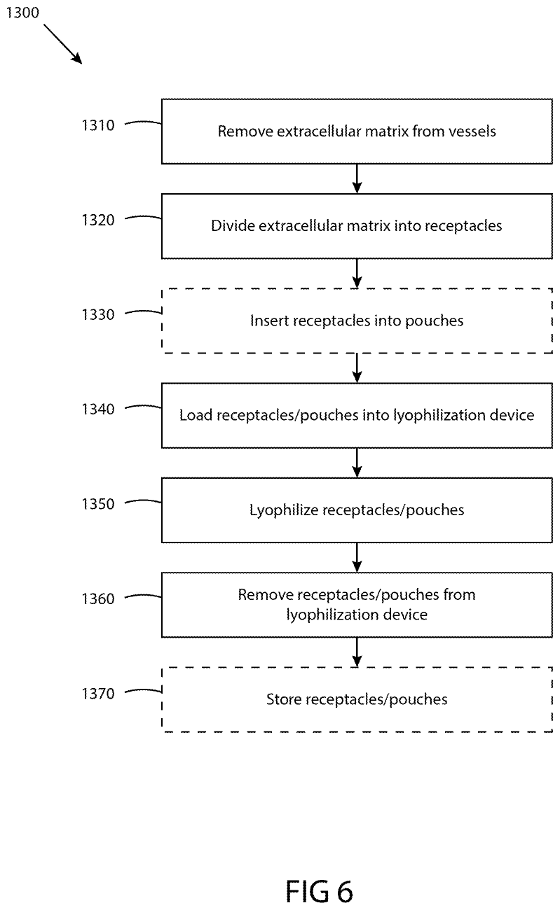

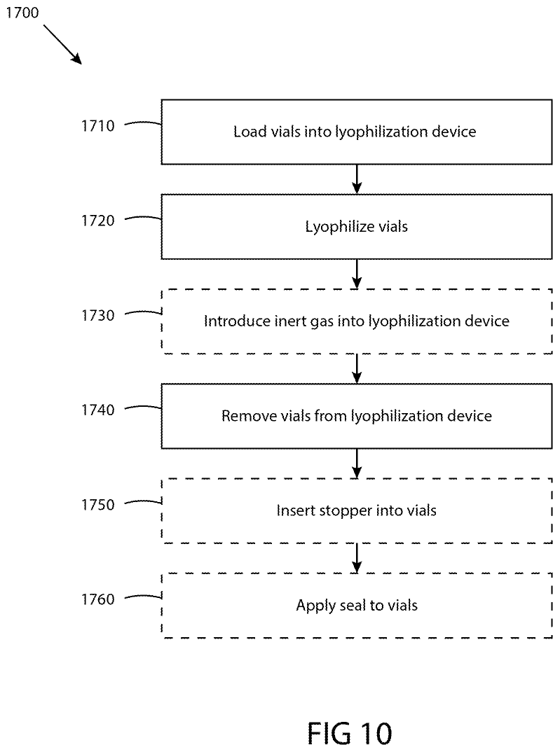

System 10 can further comprise one or more lyophilization devices, device 606 shown, such as a device configured to preserve a product (e.g. ECM 120) via a low temperature dehydration process. In some embodiments, lyophilization device 606 is configured to dehydrate the product to a residual moisture content of between 0.1% and 10%, such as a residual moisture content between 2% and 4%, such as a residual moisture content of less than 4% (e.g. the moisture content as measured via the Karl-Fischer moisture content test). In some embodiments, lyophilization device 606 is configured to dehydrate the product to a residual moisture content of between 0.2% and 2.5%. The low temperature dehydration process executed by lyophilization device 606 can comprise three primary phases: freezing, primary drying (e.g. sublimation), and secondary drying (adsorption). First, the freezing phase can be configured to cool the product within lyophilization device 606 to a temperature below its triple point to ensure sublimation, thereby preserving the product's physical form. Secondly, the primary drying phase can be configured to lower the pressure within lyophilization device 606 and can be configured to heat the product to a temperature configured to promote water sublimation. Finally, the secondary drying phase can be configured to further heat the product to a temperature configured to remove ionically-bound water molecules (e.g. break the bonds between the product and the water molecules). In some embodiments, the low temperature dehydration process comprises Methods 1300, 1700 described herein in reference to FIGS. 6, 10, respectively.

System 10 can further comprise one or more lyophilization receptacles, receptacle 607 shown, which can be configured for use with lyophilization device 606 described herein. Receptacle 607 can be configured to receive a product (e.g. ECM 120) and can be placed within lyophilization device 606 for the duration of the dehydration process. Receptacle 607 can comprise a material selected from the group consisting of: aluminum; stainless steel; glass; plastic; and combinations of these. Additionally, receptacle 607 can be depyrogenated, such as to prevent contamination of the product from pathogens on receptacle 607. In some embodiments, receptacle 607 is inserted into a lyophilization pouch 614, as described herein, prior to its placement within lyophilization device 606.

System 10 can further comprise one or more tubes, tube 608 shown, which can be configured to store an object (e.g. ECM 120). Tube 608 can include a top, or other moveable cover.

System 10 can further comprise one or more batch mills, mill 609 shown, which can be configured to grind soft, fibrous, and/or brittle products (e.g. ECM 120). Batch mill 609 can be configured to receive tube 608, as described herein, and grind the product within tube 608. In some embodiments, the products (e.g. ECM 120) are first frozen and/or maintained in a frozen state via liquid nitrogen and/or dry ice, such that the products (e.g. ECM 120) are cryogenically ground.

System 10 can further comprise one or more containers, bottle 610 shown, which can be configured to store one, two, or more fluids, powders, capsules, and the like. Bottle 610 can include a top, or other moveable cover. Bottle 610 can comprise a material selected from the group consisting of: glass; plastic, such as polycarbonate, polypropylene, polyethylene, other polyolefins, cyclic olefin copolymer; metal, such as stainless steel; and combinations of these. In some embodiments, bottle 610 comprises a volume of between 0.1 L and 5 L, such as a volume of approximately 1 L.

System 10 can further comprise one or more secondary packaging, packaging 611 shown, which can be configured to store one, two, or more other components of system 10, such as vial 210 and/or syringe 220. Packaging 611 can comprise a configuration selected from the group consisting of: envelope; card; tray; pouch; tube; bag; box; crate; drum; and combinations of these. Packaging 611 can comprise one or more materials that are impermeable to fluid. In some embodiments, a vacuum source is applied to packaging 611 to create a seal, such as to prevent or otherwise reduce a fluid or air from entering packaging 611 during storage and/or transportation. In some embodiments, heat is applied to packaging 611 to create a seal, such as to prevent or otherwise reduce a fluid or air from entering packaging 611. Packaging 611 can comprise a material selected from the group consisting of: PETG (Polyethylene Terephthalate Glycol); APET (Amorphous Polyethylene Terephthalate); HIPS (High impact Polystyrene); PVC (Polyvinyl chloride); PP (polypropylene); HDPE (High density polyethylene); PC (polycarbonate); recycled PET (polyethylene terephthalate); and combinations of these.

System 10 can further comprise one or more tertiary packaging, packaging 612 shown, which can be configured to store one, two, or more other components of system 10, such as secondary packaging 611. Packaging 612 can comprise a configuration selected from the group consisting of: envelope; pouch; tube; bag; box; crate; drum; and combinations of these.

System 10 can further comprise one or more sterilization chambers, chamber 613 shown. Sterilization chamber 613 can be configured to eliminate, remove, kill, or deactivate biological agents (e.g. bacteria, viruses, etc.) on an object (e.g. vials 210). Sterilization chamber 613 can be configured to implement a sterilization method selected from the group consisting of: heat, such as dry heat, steam; chemical, such as ethylene oxide, peracetic acid; irradiation, such as electron beam processing, gamma radiation; high pressure, such as pascalization; filtration, such as microfiltration; and combinations of these. System 10 can further comprise one or more lyophilization pouches, pouch 614 shown, which can be configured for use with lyophilization device 606 described herein. Pouch 614 can be configured to receive a product (e.g. receptacle 607, EMC 120) and can be placed within lyophilization device 606 for the duration of the dehydration process. Pouch 614 can be configured as permeable to fluid (e.g. water) but impermeable to contaminants (e.g. germs, dust particles, etc.). Pouch 614 can comprise a material selected from the group consisting of: polyethylene, such as Tyvek®; medical grade paper; foil, such as aluminum foil; and combinations of these.

System 10 can further comprise one or more buffer solutions, solution 701 shown, which can be configured to resist changes in pH when an acid and/or alkali is added to it (e.g. maintain a constant pH). In some embodiments, buffer solution 701 comprises a phosphate-buffered solution or phosphate-buffered saline (PBS).

System 10 can further comprise one or more cooling agents, agent 702 shown, which can be configured to reduce, and/or otherwise regulate, the temperature of a product (e.g. raw material 65). Cooling agent 702 can comprise an agent selected from the group consisting of: dry ice; dry ice with ethanol; dry ice with acetone; liquid nitrogen; wet ice; frozen ice packs; cold packs, and combinations of these.

System 10 can further comprise one or more purified waters, water 703 shown, which can comprise water that has been filtered, or otherwise processed, to remove one, two, or more impurities. In some embodiments, purified water 703 comprises Type I water or water for injection.

System 10 can further comprise one or more dissociation solutions, solution 704 shown, which can be configured to dissociate adherent cells, cell aggregates, and/or tissues into single-cell suspensions. In some embodiments, dissociation solution 704 comprises a co-solution comprising 0.02% (w/v) trypsin and a range between 0.008% and 0.05% (w/v) of ethylenediaminetetraacetic acid (EDTA). Dissociation solution 704 can comprise a solution that is warmed to a temperature of approximately 35° C.

System 10 can further comprise one or more disinfecting solutions, solution 705 shown, which can be configured to destroy one, two, or more microorganisms (e.g. bacteria, virus, fungi). In some embodiments, disinfecting solution 705 comprises a co-solution comprising 0.1% (v/v) peracetic acid and 4% (v/v) ethanol.

System 10 can further comprise one or more detergent solutions, solution 706 shown, which can be configured to lyse and/or permeabilize cells. In some embodiments, detergent solution 706 comprises a 3% (v/v) Triton X-100 solution. In some embodiments, detergent solution 706 comprises a 4% (w/v) sodium deoxycholate solution.

System 10 can further comprise one or more sucrose solutions, solution 707 shown, which can be configured as an excipient. Sucrose solution 707 can stabilize biological material (e.g. raw material 65, ECM 120). Sucrose solution 707 can provide cryoprotection and/or lyoprotectant to biological material (e.g. raw material 65, ECM 120). In some embodiment, sucrose solution 707 comprises a 1M sucrose solution.

System 10 can further comprise one or more sterile waters, water 708 shown, which can comprise water that has been processed to remove one, two, or more contaminants (e.g. bacteria, virus, fungi). In some embodiments, sterile water 708 comprises water for injection.

System 10 can further comprise one or more acid solutions, solution 709 shown, which can be configured to solubilize, degrade, and/or disinfect tissue. In some embodiments, acid solution 709 comprises a 0.01 N hydrochloric acid (HCl) solution.

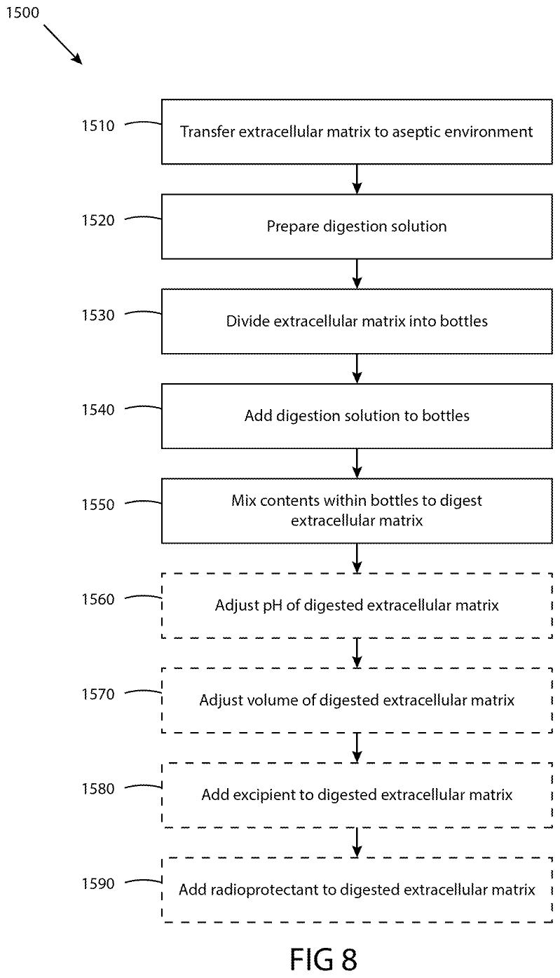

System 10 can further comprise one or more digestive enzymes, enzyme 710 shown, which can be configured to break down macromolecules. In some embodiments, the digestive enzyme comprises pepsin comprising an activity level of between 0.5 U/mg and 5000 U/mg, such as an activity level of approximately 250 U/mg, or such as an activity level of approximately 2500 U/mg. As described herein in reference to STEP 1520 of FIG. 8, enzyme 710 can be added to acid solution 709 such that the final concentration results in an activity level of between 10 U/mL and 2500 U/mL, such as an activity level of 250 U/mg. Enzyme 710 can comprise a concentration configured to alter (e.g. increase, decrease) the gel mechanical properties and/or the gelation kinetics of ECM 120.

ECM 120 treated with enzyme 710 can be reconstituted and neutralized with PBS comprising a concentration/ionic strength equivalent to 75% of that of an isotonic solution. In some embodiments, enzyme 710 comprises a concentration of approximately 100 U/mL and is configured to result in an ECM 120 comprising a storage modulus of no less than 25 Pa and no more than 40 Pa. In some embodiments, enzyme 710 comprises a concentration of approximately 250 U/mL and is configured to result in an ECM 120 comprising a storage modulus of no less than 90 Pa and no more than 130 Pa. In some embodiments, enzyme 710 comprises a concentration of approximately 500 U/mL and is configured to result in an ECM 120 comprising a storage modulus of no less than 70 Pa and no more than 105 Pa. In some embodiments, enzyme 710 comprises a concentration of approximately 1000 U/mL and is configured to result in an ECM 120 comprising a storage modulus of no less than 85 Pa and no more than 130 Pa.

ECM 120 treated with enzyme 710 can be reconstituted and neutralized with PBS comprising a concentration/ionic strength equivalent to 62.5% of that of an isotonic solution. In some embodiments, enzyme 710 comprises a concentration of approximately 100 U/mL and is configured to result in an ECM 120 comprising a storage modulus of no less than 30 Pa and no more than 45 Pa. In some embodiments, enzyme 710 comprises a concentration of approximately 250 U/mL and is configured to result in an ECM 120 comprising a storage modulus of no less than 90 Pa and no more than 140 Pa. In some embodiments, enzyme 710 comprises a concentration of approximately 500 U/mL and is configured to result in an ECM 120 comprising a storage modulus of no less than 85 Pa and no more than 125 Pa. In some embodiments, enzyme 710 comprises a concentration of approximately 1000 U/mL and is configured to result in an ECM 120 comprising a storage modulus of no less than 100 Pa and no more than 155 Pa.

ECM 120 treated with enzyme 710 can be reconstituted and neutralized with PBS comprising a concentration/ionic strength equivalent to 50% of that of an isotonic solution. In some embodiments, enzyme 710 comprises a concentration of approximately 100 U/mL and is configured to result in an ECM 120 comprising a storage modulus of no less than 45 Pa and no more than 75 Pa. In some embodiments, enzyme 710 comprises a concentration of approximately 250 U/mL and is configured to result in an ECM 120 comprising a storage modulus of no less than 155 Pa and no more than 220 Pa. In some embodiments, enzyme 710 comprises a concentration of approximately 500 U/mL and is configured to result in an ECM 120 comprising a storage modulus of no less than 120 Pa and no more than 190 Pa. In some embodiments, enzyme 710 comprises a concentration of approximately 1000 U/mL and is configured to result in an ECM 120 comprising a storage modulus of no less than 120 Pa and no more than 180 Pa.

Alternatively or additionally, enzyme 710 can comprise a concentration configured to alter (e.g. increase, decrease) the shelf life of ECM 120 at a storage temperature of between 2° C. and 25° C. In some embodiments, enzyme 710 comprises a concentration of approximately 100 U/mL and ECM 120 including enzyme 710 comprises a shelf life of more than three months at a temperature of between 20° C. and 25° C., such as a temperature of 22° C. (e.g. room temperature). In some embodiments, enzyme 710 comprises a concentration of approximately 1000 U/mL and ECM 120 including enzyme 710 comprises a shelf life of no more than one month at a temperature of between 20° C. and 25° C., such as a temperature of 22° C. (e.g. room temperature).

System 10 can further comprise one or more excipients, excipient 711 shown, which can be configured to provide at least one of long-term stabilization, bulking, radioprotection, heat protection, cryoprotection, lyoprotection, increase in solubility, or other enhancement of a product. Excipient 711 can comprise an excipient selected from the group consisting of: sucrose; ascorbic acid; sodium ascorbate; sodium azide; Vitamin E; ethylenediaminetetraacetic acid (EDTA); mannitol; glycerol, and combinations of these. Excipient 711 can be configured to increase, and/or otherwise improve, the relative solubility of a product (e.g. ECM 120). Excipient 711 can be configured to increase, or otherwise improve, the relative gelation of a product (e.g. ECM 120).

System 10 can further comprise one or more radioprotective agents, radioprotectant 712 shown, which can be configured to reduce free radical damage of a material (e.g. ECM 120) exposed to ionizing radiation. Radioprotectant 712 can be configured to prevent or otherwise reduce the scissioning of peptides during irradiation-based sterilization methods (e.g. e-beam, gamma, x-ray) without cytotoxic effects following implantation into a patient. Radioprotectant 712 can comprise an agent selected from the group consisting of: vitamin E and/or vitamin E derivatives (e.g. alpha-, beta-, gamma-, delta-tocopherol and tocotrienol, tocopherol acetate; chromanol-alpha-C6; 6-hydroxy-2,5,7,8-tetramethylchroma-2 carboxylic acid (Trolox), dl-a-tocopherol (Synthetic). D-alpha-Tocopheryl polyethylene glycol succinate (TPGS), Vitamin E Succinate) comprising a concentration between 0.01 mg/mL and 50 mg/mL, such as between 0.2 mg/mL and 10 mg/mL; ascorbic acid (e.g. Vitamin C) comprising a concentration of between 0.01 mg/mL and 50 mg/mL, such as between 0.2 mg/mL and 10 mg/mL, such as between 0.35 mg/mL and 3.5 mg/mL; glycerol comprising a concentration of between 0.1 mg/mL and 10 mg/mL, such as between 0.2 mg/mL and 5 mg/mL, such as between 0.5 mg/mL and 2 mg/mL; riboflavin (e.g. Vitamin B2) comprising a concentration between 0.05 mg/mL and 10 mg/mL, such as between 0.1 mg/mL and 5 mg/mL, such as between 0.1 mg/mL and 1 mg/mL; polyvinylpyrrolidone (PVP) comprising a concentration between 0.05 mg/mL and 10 mg/mL, such as between 0.1 mg/mL and 5 mg/mL, such as between 0.1 mg/mL and 1.5 mg/mL; sodium ascorbate comprising a concentration between 0.005 mg/mL and 40 mg/mL, such as between 0.05 mg/mL and 4 mg/mL; sodium azide comprising a concentration between 0.03 mg/mL and 15 mg/mL, such as between 0.3 mg/mL and 1 mg/mL; hydroquinone comprising a concentration between 0.2 mg/mL and 35 mg/mL, such as 2.0 mg/mL and 5.0 mg/mL; and combinations of these.

Referring now to FIG. 2, a perspective view of a medical device comprising a conduit is illustrated, consistent with the present inventive concepts. Implant 20 comprises a conduit (e.g. artificial, natural, or combinations of these) configured to connect, or otherwise provide one, two, or more channels, between two or more anatomical elements (e.g. nerve stumps, nerve fascicles, etc.). Implant 20 can comprise at least a first end 21 and at least a second end 23, with a lumen 22 therebetween. First end 21 can be constructed and arranged to receive at least a portion of a first anatomical element (e.g. first nerve stump, first nerve fascicles, etc.) and second end 23 can be constructed and arranged to receive at least a portion of a second anatomical element (e.g. second nerve stump).

Lumen 22 can be configured to receive, or otherwise comprise, a therapeutic device (e.g. device 100 of the present inventive concepts), such as to maintain the relative positioning of the therapeutic device between the two or more anatomical elements. Alternatively or additionally, first end 21 and/or second end 23 can be configured to receive, or otherwise comprise, a therapeutic device (e.g. device 100 of the present inventive concepts), such that the therapeutic device contacts at least a portion of the anatomical elements received by first end 21, second end 23.

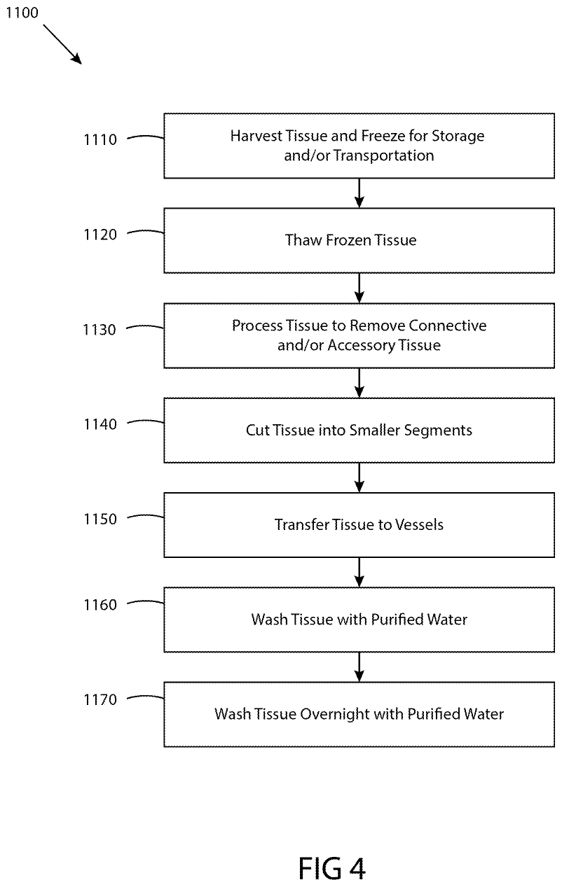

Referring now to FIG. 3, a method for producing an extracellular matrix from tissue is illustrated, consistent with the present inventive concepts. Method 1000 comprises a sequence of sub-methods, Methods 1100, 1200, 1300, 1400, 1500, 1600, 1700, 1800, 1900, 2000, 2100, 2200, 2300, 2400, 2500, and 2600 as described herein in reference to FIGS. 4-19, respectively. Method 1100 comprises a method for harvesting and/or preparing tissue for further manipulation. Method 1200 comprises a method for decellularizing the tissue harvested and/or prepared in Method 1100 to produce an extracellular matrix. Method 1300 comprises a method for lyophilizing the extracellular matrix produced in Method 1200. Method 1400 comprises a method for mechanically disrupting the extracellular matrix produced in Method 1300. Method 1400 can proceed to one of Method 1500 or 1900.

In a first embodiment, Method 1400 proceeds to Method 1500. As described herein, Methods 1500-1800 are performed within an aseptic environment and/or a pre-sterilized, semi-closed, and/or single-use system. Method 1500 comprises a method for digesting the extracellular matrix produced in Method 1400. Method 1600 comprises a method for aliquoting the extracellular matrix produced in Method 1500 between one, two, or more containers. Method 1700 comprises a method for lyophilizing the containers comprising the extracellular matrix produced in Method 1600. Method 1800 comprises a method for packaging and/or storing the containers comprising the extracellular matrix produced in Method 1700.

In a second embodiment, Method 1400 proceeds to Method 1900. Method 1900 comprises a method for digesting the extracellular matrix produced in Method 1400. Method 2000 comprises a method for aliquoting the extracellular matrix produced in Method 1900 between one, two, or more containers. Method 2000 can proceed to one of Method 2100 or 2400.

In a first embodiment, Method 2000 proceeds to Method 2100. As described herein, Methods 2100-2300 are configured to prepare the extracellular matrix for sterilization via irradiation. Method 2100 comprises a method for lyophilizing the containers comprising the extracellular matrix produced in Method 2000. Method 2200 comprises a method for packaging and/or storing the containers comprising the extracellular matrix produced in Method 2100. Method 2300 comprises a method for sterilizing the containers comprising the extracellular matrix produced in Method 2200 via irradiation.

In a second embodiment, Method 2000 proceeds to Method 2400. As described herein, Methods 2400-2600 are configured to prepare the extracellular matrix for sterilization via gas. Method 2400 comprises a method for lyophilizing the containers comprising the extracellular matrix produced in Method 2000. Method 2500 comprise a method for packaging and/or storing the containers comprising the extracellular matrix produced in Method 2400. Method 2600 comprises a method for sterilizing the containers comprising the extracellular matrix produced in Method 2500 via gas.

Referring now to FIG. 4, a method for harvesting and/or preparing tissue for further manipulation is illustrated, consistent with the present inventive concepts. Method 1100 can be configured to harvest and/or prepare raw material 65 from tissue source 60 described herein in reference to FIG. 1.

In STEP 1110, raw material 65 is harvested from a tissue source (e.g. tissue source 60). Additionally, raw material 65 can be processed to remove connective and/or accessory tissue (e.g. remove non-nerve tissue). For short-term storage (e.g. for a duration less than six hours), cleaned raw material 65 can be at least partially immersed in buffer solution 701. In some embodiments, raw material 65 can be stored in chamber 601 at a temperature between approximately 2° C. and 8° C. For long-term storage and/or transportation (e.g. for a duration more than six hours), raw material 65 can be rapidly frozen in buffer solution 701. In some embodiments, raw material 65 is rapidly frozen via cooling agent 702. Raw material 65 can be stored and/or transported in chamber 601 at a temperature of approximately −80° C. (or lower temperatures such as those afforded by dry ice or liquid nitrogen storage). In some embodiments, raw material 65 is stored in chamber 601 at a temperature of approximately −80° C. for a maximum of six months.

In STEP 1120, frozen raw material 65 is thawed in chamber 601 at a temperature of between 2° C. and 8° C. In some embodiments, raw material 65 is thawed in chamber 601 for at least 48 hours, such as at least 72 hours.

In STEP 1130, raw material 65 is further processed (e.g. cleaned) to remove additional connective and/or accessory tissue (e.g. remove non-nerve tissue). Raw material 65 can be processed at a temperature of between 2° C. and 25° C. In some embodiments, cleaned raw material 65 comprises a final mass:initial mass ratio of at least 1:2 (e.g. connective and/or accessory tissue removed during processing comprises less than 50% of the initial mass).

In STEP 1140, cleaned raw material 65 is cut, or otherwise divided, into smaller segments. Cleaned raw material 65 can be cut into segments between 0.5 cm and 2 cm, such as 1 cm segments.

In STEP 1150, raw material 65 is transferred to one, two, or more vessels 602. Cleaned raw material 65 can be transferred at a temperature of between 2° C. and 25° C. In some embodiments, each vessel 602 comprises no more than 25 g of cleaned raw material 65 and a mixing device 603 contains no more than six vessels 602.

In STEP 1160, cleaned raw material 65 is washed with purified water 703. Cleaned raw material 65 can be washed at a temperature of between 2° C. and 8° C. Cleaned raw material 65 is washed with purified water 703 at least two times, such as at least three times, such as at least four times. Cleaned raw material 65 and purified water 703 can comprise a ratio between 1:20 and 1:50, such as 1:30. In some embodiments, vessel 602 is placed into mixing device 603 comprising purified water 703, such as a mixing device comprising at least 3000 mL of purified water 703. Mixing device 603 is placed on top of heating device 604 configured to stir purified water 703 at a speed between 10 rpm and 1000 rpm, such as 100±10 rpm, for at least 10 minutes, thereby washing cleaned raw material 65 within vessel 602. Purified water 703 is decanted from mixing device 603 and replaced with fresh purified water 703. Mixing device 603 is placed back on top of heating device 604 configured to stir purified water 703 at a speed between 10 rpm and 1000 rpm, such as 100±10 rpm, for at least 10 minutes, thereby washing raw material 65 within vessel 602 a second time. Purified water 703 is decanted from mixing device 603.

In STEP 1170, cleaned raw material 65 is washed overnight with purified water 703. Cleaned raw material 65 can be washed at a temperature of between 2° C. and 8° C. Cleaned raw material 65 and purified water 703 can comprise a ratio between 1:20 and 1:50, such as 1:30. In some embodiments, vessel 602 is placed into mixing device 603 comprising purified water 703, such as a mixing device comprising at least 3000 mL of purified water 703. Mixing device 603 is stored in chamber 601 at a temperature of approximately 5° C. for between 12 hours and 24 hours, such as 16 hours. During this time, mixing device 603 is placed on top of heating device 604 configured to stir purified water 703 at speed between 10 rpm and 1000 rpm, such as 100±10 rpm, thereby washing cleaned raw material 65 within vessel 602.

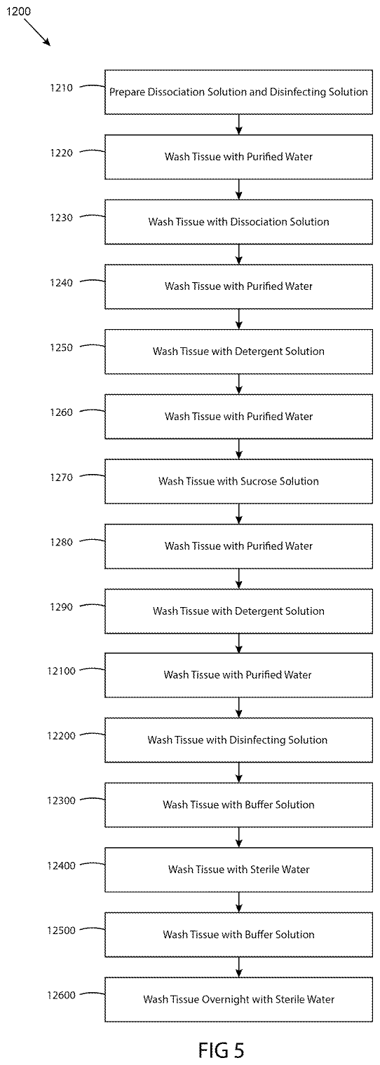

Referring now to FIG. 5, a method for decellularizing tissue to produce an extracellular matrix is illustrated, consistent with the present inventive concepts. Method 1200 can be configured to decellularize cleaned raw material 65 harvested and/or prepared in Method 1100 described herein in reference to FIG. 4.

In STEP 1210, dissociation solution 704 and disinfecting solution 705 are prepared. Dissociation solution 704 and disinfecting solution 705 can be prepared at a temperature of between 2° C. and 25° C.

In STEP 1220, cleaned raw material 65 from STEP 1170 is washed with purified water 703. In some embodiments, purified water 703 is pre-chilled in chamber 601 to a temperature of between 2° C. and 8° C. Cleaned raw material 65 is washed with purified water 703 at least two times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of approximately 5° C. Mixing device 603 is placed on top of heating device 604 configured to stir purified water 703 at 100±10 rpm, for at least 10 minutes, thereby washing cleaned raw material 65 within vessel 602. Purified water 703 is decanted from mixing device 603 and replaced with fresh purified water 703. Mixing device 603 is placed back on top of heating device 604 configured to stir purified water 703 at a speed between 10 rpm and 1000 rpm, such as 100±10 rpm, for at least 10 minutes, thereby washing cleaned raw material 65 within vessel 602 a second time. Purified water 703 is decanted from mixing device 603.

In STEP 1230, cleaned raw material 65 is washed with dissociation solution 704. Dissociation solution 704 can comprise a temperature of between 2° C. and 37° C., such as 35±2° C. Cleaned raw material 65 and dissociation solution 704 can comprise a ratio between 1:20 and 1:50, such as 1:30. Dissociation solution 704 is added to mixing device 603. In some embodiments, at least 3000 mL of dissociation solution 704 is added to mixing device 603. Mixing device 603 is placed on top of heating device 604 configured to stir dissociation solution 704 at a speed between 10 rpm and 1000 rpm, such as 100±10 rpm, thereby washing cleaned raw material 65 within vessel 602. Cleaned raw material 65 is washed in chamber 601 at a temperature of between 2° C. and 37° C., such as 35±2° C., for between 30 minutes and 180 minutes, such as 60±5 minutes. In some embodiments, a lipid layer forms on the surface of dissociation solution 704 and is removed using instrument 605. Dissociation solution 704 is decanted from mixing device 603.

In STEP 1240, cleaned raw material 65 is washed with purified water 703. In some embodiments, purified water 703 is pre-chilled in chamber 601 to a temperature of between 2° C. and 8° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of approximately 5° C. Mixing device 603 is placed on top of heating device 604 configured to stir purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 1250, cleaned raw material 65 is washed with detergent solution 706. In some embodiments, detergent solution 706 is pre-chilled in chamber 601 to a temperature of between 2° C. and 8° C. Cleaned raw material 65 and detergent solution 706 can comprise a ratio between 1:20 and 1:50, such as 1:30. Detergent solution 706 is added to mixing device 603. In some embodiments, at least 3000 mL of detergent solution 706 is added to mixing device 603. Mixing device 603 is stored in chamber 601 at a temperature of between 2° C. and 8° C., such as approximately 4° C. Mixing device 603 is placed on top of heating device 604 configured to stir detergent solution 706 at a speed between 10 rpm and 1000 rpm, such as 100±10 rpm, for between 30 minutes and 180 minutes, such as 60±5 minutes, thereby washing cleaned raw material 65 within vessel 602. Detergent solution 706 is decanted from mixing device 603.

In STEP 1260, cleaned raw material 65 is washed with purified water 703. In some embodiments, purified water 703 is pre-chilled in chamber 601 to a temperature of between 2° C. and 8° C., such as approximately 4° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 8° C., such as approximately 4° C. Mixing device 603 is placed on top of heating device 604 configured to stir purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 1270, cleaned raw material 65 is washed with sucrose solution 707. In some embodiments, sucrose solution 707 is pre-chilled in chamber 601 to a temperature of between 2° C. and 8° C., such as approximately 4° C. Cleaned raw material 65 and sucrose solution 707 can comprise a ratio between 1:20 and 1:50, such as 1:30. Sucrose solution 707 is added to mixing device 603. In some embodiments, at least 3000 mL of sucrose solution 707 is added to mixing device 603. Mixing device 603 is stored in chamber 601 at a temperature of between 2° C. and 8° C., such as approximately 4° C. Mixing device 603 is placed on top of heating device 604 configured to stir sucrose solution 707 at a speed between 10 rpm and 1000 rpm, such as 100±10 rpm, for between 5 minutes and 60 minutes, such as 15±5 minutes, thereby washing cleaned raw material 65 within vessel 602. Sucrose solution 707 is decanted from mixing device 603.

In STEP 1280, cleaned raw material 65 is washed with purified water 703. In some embodiments, purified water 703 is pre-chilled in chamber 601 to a temperature of between 2° C. and 8° C., such as approximately 4° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 8° C., such as approximately 4° C. Mixing device 603 is placed on top of heating device 604 configured to stir purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 1290, cleaned raw material 65 is washed with detergent solution 706. In some embodiments, detergent solution 706 is pre-chilled in chamber 601 to a temperature of between 2° C. and 8° C., such as approximately 4° C. Cleaned raw material 65 and detergent solution 706 can comprise a ratio between 1:20 and 1:50, such as 1:30. Detergent solution 706 is added to mixing device 603. In some embodiments, at least 3000 mL of detergent solution 706 is added to mixing device 603. Mixing device 603 is stored in chamber 601 at a temperature of between 2° C. and 8° C., such as approximately 4° C. Mixing device 603 is placed on top of heating device 604 configured to stir detergent solution 706 at a speed between 10 rpm and 1000 rpm, such as 100±10 rpm, for between 30 minutes and 180 minutes, such as 60±5 minutes, thereby washing cleaned raw material 65 within vessel 602. Detergent solution 706 is decanted from mixing device 603.

In STEP 12100, cleaned raw material 65 is washed with purified water 703. In some embodiments, purified water 703 is pre-chilled in chamber 601 to a temperature of between 2° C. and 8° C., such as approximately 4° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 8° C., such as approximately 4° C. Mixing device 603 is placed on top of heating device 604 configured to stir purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 12200, cleaned raw material 65 is washed with disinfecting solution 705. In some embodiments, disinfecting solution 705 is pre-chilled in chamber 601 to a temperature of between 2° C. and 8° C., such as approximately 4° C. Cleaned raw material 65 and disinfecting solution 705 can comprise a ratio between 1:20 and 1:50, such as 1:30. Disinfecting solution 705 is added to mixing device 603. In some embodiments, at least 3000 mL of disinfecting solution 705 is added to mixing device 603. Mixing device 603 is stored in chamber 601 at a temperature of between 2° C. and 8° C., such as approximately 4° C. Mixing device 603 is placed on top of heating device 604 configured to stir disinfecting solution 705 at a speed between 10 rpm and 1000 rpm, such as 100±10 rpm, for between 30 minutes and 240 minutes, such as 120±5 minutes, thereby washing cleaned raw material 65 within vessel 602. Disinfecting solution 705 is decanted from mixing device 603.

In STEP 12300, cleaned raw material 65 is washed with buffer solution 701. In some embodiments, buffer solution 701 is pre-chilled in chamber 601 to a temperature of between 2° C. and 8° C., such as approximately 4° C. Raw material 65 and buffer solution 701 can comprise a ratio between 1:20 and 1:50, such as 1:30. Buffer solution 701 is added to mixing device 603. In some embodiments, at least 3000 mL of buffer solution 701 is added to mixing device 603. Mixing device 603 is stored in chamber 601 at a temperature of between 2° C. and 8° C., such as approximately 4° C. Mixing device 603 is placed on top of heating device 604 configured to stir buffer solution 701 at speed between 10 rpm and 1000 rpm, such as 100±10 rpm, for between 5 minutes and 60 minutes, such as 15±5 minutes, thereby washing cleaned raw material 65 within vessel 602. Buffer solution 701 is decanted from mixing device 603.

In STEP 12400, cleaned raw material 65 is washed with sterile water 708. In some embodiments, sterile water 708 is pre-chilled in chamber 601 to a temperature of between 2° C. and 8° C., such as approximately 4° C. Cleaned raw material 65 is washed with sterile water 708 at least two times. Cleaned raw material 65 and sterile water 708 can comprise a ratio between 1:20 and 1:50, such as 1:30. Sterile water 708 is added to mixing device 603. In some embodiments, at least 3000 mL of sterile water 708 is added to mixing device 603. Mixing device 603 is stored in chamber 601 at a temperature of between 2° C. and 8° C., such as approximately 4° C. Mixing device 603 is placed on top of heating device 604 configured to stir sterile water solution at a speed between 10 rpm and 1000 rpm, such as 100±10 rpm, for between 5 minutes and 60 minutes, such as 15±5 minutes, thereby washing cleaned raw material 65 within vessel 602. Sterile water 708 is decanted from mixing device 603 and replaced with fresh sterile water solution. Mixing device 603 is placed back on top of heating device 604 configured to stir sterile water 708 at a speed between 10 rpm and 1000 rpm, such as 100±10 rpm, for between 5 minutes and 60 minutes, such as 15±5 minutes, thereby washing cleaned raw material 65 within vessel 602 a second time. Sterile water 708 is decanted from mixing device 603.

In STEP 12500, cleaned raw material 65 is washed with buffer solution. In some embodiments, buffer solution 701 is pre-chilled in chamber 601 to a temperature of between 2° C. and 8° C., such as approximately 4° C. Cleaned raw material 65 and buffer solution 701 can comprise a ratio between 1:20 and 1:50, such as 1:30. Buffer solution 701 is added to mixing device 603. In some embodiments, at least 3000 mL of buffer solution 701 is added to mixing device 603. Mixing device 603 is stored in chamber 601 at a temperature of between 2° C. and 8° C., such as approximately 4° C. Mixing device 603 is placed on top of heating device 604 configured to stir buffer solution 701 at a speed between 10 rpm and 1000 rpm, such as 100±10 rpm, for between 5 minutes and 60 minutes, such as 15±5 minutes, thereby washing cleaned raw material 65 within vessel 602. Buffer solution 701 is decanted from mixing device 603.

In STEP 12600, cleaned raw material 65 is washed overnight with sterile water. In some embodiments, sterile water 708 is pre-chilled in chamber 601 to a temperature of between 2° C. and 8° C., such as approximately 4° C. Cleaned raw material 65 and sterile water 708 can comprise a ratio between 1:20 and 1:50, such as 1:30. Sterile water 708 is added to mixing device 603. In some embodiments, at least 3000 mL of sterile water 708 is added to mixing device 603. Mixing device 603 is stored in chamber 601 at a temperature of between 2° C. and 8° C., such as approximately 4° C., for between 12 hours and 24 hours, such as 16±2 hours. During this time, mixing device 603 is placed on top of heating device 604 configured to stir purified water 703 at a speed between 10 rpm and 1000 rpm, such as 100±10 rpm, thereby washing cleaned raw material 65 within vessel 602. Sterile water 708 is decanted from mixing device 603.

Upon the conclusion of Method 1200, cleaned raw material 65 comprises a decellularized extracellular matrix (referred to as “raw ECM 120” herein).

Referring now to FIG. 6, a method for lyophilizing an extracellular matrix is illustrated, consistent with the present inventive concepts. Method 1300 can be configured to lyophilize raw ECM 120 produced in Method 1200 described herein in reference to FIG. 5.

In STEP 1310, raw ECM 120 is removed from vessel 602. In some embodiments,

In STEP 1320, raw ECM 120 is divided and transferred into one, two, or more lyophilization receptacles 607. Raw ECM 120 can be manually transferred via a depyrogenated instrument 605, such as to prevent contamination of raw ECM 120 from pathogens on the instrument.

In STEP 1330, comprising an optional step, receptacles 607 comprising raw ECM 120 can be inserted into a lyophilization pouch 614. In some embodiments, one, two, three or more receptacles 607 are inserted into a single pouch 614.

In STEP 1340, receptacles 607 and/or pouches 614 comprising raw ECM 120 are loaded into lyophilization device 606. In some embodiments, receptacles 607 and/or pouches 614 are loaded into a preconditioned lyophilization device 606.

In STEP 1350, receptacles 607 and/or pouches 614 comprising raw ECM 120 are lyophilized via lyophilization device 606. In some embodiments, lyophilization device 606 can be configured to freeze receptacles 607 and/or pouches 614 at a temperature of approximately −40° C. for no less than four hours. In some embodiments, lyophilization device 606 can be configured to apply a vacuum source to receptacles 607 and/or pouches 614. In some embodiments, the vacuum source comprises 150 micrometers of Hg. In some embodiments, lyophilization device 606 can be configured to dry receptacles 607 and/or pouches 614 at a temperature of between −8° C. and 0° C., increasing the temperature over time. In some embodiments, lyophilization device 606 can be configured to increase the temperature to between 20° C. and 25° C., such as a temperature of 22° C. (e.g. room temperature). In some embodiments, the total cycle duration can be configured to comprise a duration of between 12 and 66 hours, such as a duration between 18 and 24 hours, such as approximately 24 hours.

In STEP 1360, receptacles 607 and/or pouches 614 comprising raw ECM 120 are removed from lyophilization device 606.

In STEP 1370, comprising an optional step, receptacles 607 and/or pouches 614 comprising raw ECM 120 can be stored in chamber 601 at temperature of approximately −80° C. Upon the conclusion of Method 1300, raw ECM 120 comprises a lyophilized decellularized extracellular matrix (referred to as “lyophilized ECM 120” herein).

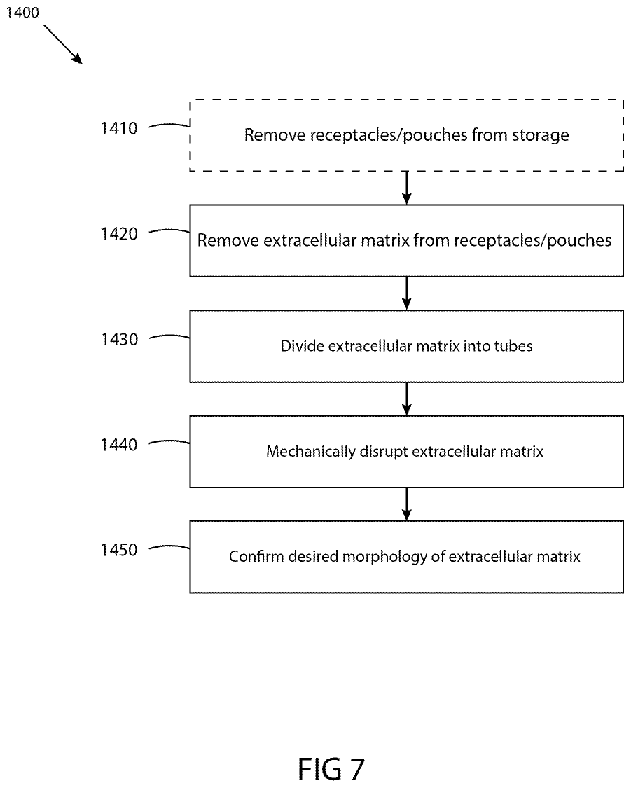

Referring now to FIG. 7, a method for mechanically disrupting an extracellular matrix is illustrated, consistent with the present inventive concepts. Method 1400 can be configured to grind lyophilized ECM 120 produced in Method 1300 described herein in reference to FIG. 6.

In STEP 1410, comprising an optional step, receptacles 607 and/or pouches 614 comprising raw ECM 120 can be removed from chamber 601 and thawed.

In STEP 1420, lyophilized ECM 120 is removed from receptacles 607. In some embodiments, receptacles 607 are removed from pouches 614 prior to the removal of ECM 120.

In STEP 1430, lyophilized ECM 120 is divided and transferred into one, two, or more tubes 608. In some embodiments, approximately 10±1 g of lyophilized ECM 120 is added to tube 608. Tube 608 is closed and transferred to batch mill 609.

In STEP 1440, lyophilized ECM 120 is ground or otherwise mechanically disrupted, such as via batch mill 609. Batch mill 609 can comprise a grinding speed between 5000 rpm and 50,000 rpm, such as 25,000 rpm. Lyophilized ECM 120 can be ground in time intervals of between five seconds and 60 seconds, such as intervals of 15 seconds. Lyophilized ECM 120 is ground until a desired morphology is achieved, such as after between one and five grinding time intervals.

In STEP 1450, desired morphology of lyophilized ECM 120 is confirmed. In some embodiments, lyophilized ECM 120 is ground until it demonstrates a fiber-like morphology. In some embodiments, the desired morphology of lyophilized ECM 120 is further refined to comprise particulate of a defined size using a sieve and/or other size exclusion method.

Upon the conclusion of Method 1400, lyophilized ECM 120 comprises a ground (e.g. disrupted) decellularized extracellular matrix (referred to as “ground ECM 120” herein).

Referring now to FIG. 8, a method for digesting an extracellular matrix is illustrated, consistent with the present inventive concepts. Method 1500 can be configured to digest ground ECM 120 produced in Method 1400 described herein in reference to FIG. 7. Method 1500 is configured to be performed within an environment suitable for aseptic processing, such that that materials, devices, and components utilized in Method 1500 are transferred to and/or contained within an environment suitable for aseptic processing. In some embodiments, the materials, devices, and/or components utilized in Method 1500 comprise a pre-sterilized, semi-closed, and/or single-use system. For example, Method 1500 is performed utilizing a sterile work area and/or sterile handling, such as to prevent or otherwise reduce contamination from microorganisms (e.g. bacteria, virus, fungi).

In STEP 1510, ground ECM 120 is transferred to an environment suitable for aseptic processing. As described herein, STEPs 1520-1590 are performed within the aseptic environment.

In STEP 1520, acid solution 709 is prepared and further combined with digestive enzyme 710 (collectively “digestion solution” herein).

In STEP 1530, ground ECM 120 is divided into one, two, or more bottles 610. In some embodiments, between 1 g and 20 g, such as 8.4 g, of ground ECM 120 is added to each bottle 610. In some embodiments, bottles 610 are sterilized prior to receiving ground ECM 120.

In STEP 1540, the digestion solution from STEP 1520 is added to each bottle 610 comprising ground ECM 120. In some embodiments, between 250 mL and 1000 mL, such as 900 mL, of the digestion solution is added to each bottle 610. In some embodiments, the collective volume of ground ECM 120 and the digestion solution comprises greater than or equal to 70% of the total volume of bottle 610. In some embodiments, the final concentration of ground ECM 120 in the digestion solution is between 0.5 mg/mL and 100 mg/mL, such as 10 mg/mL.

In STEP 1550, each bottle 610 comprising ground ECM 120 and the digestion solution (collectively “digest” herein) is stored in chamber 601 at a temperature of between 2° C. and 37° C., such as at a temperature between 15° C. and 30° C., such as at a temperature between 18° C. and 23° C., and for a duration of at least 12 hours, such as for a duration between 46 and 50 hours. Mixing device 603 is lowered into each bottle 610. Mixing device 603 is configured to stir the digest at a speed between 100 rpm and 5000 rpm, such as between 700 rpm and 1400 rpm. In some embodiments, mixing device 603 is positioned between ⅓ and ½ of the height of bottle 610, such as to promote, or otherwise improve, the homogeneity of the digestion process.

For example, the speed of mixing device 603 begins at 700 rpm. After at least 90 minutes, the speed of mixing device is slowly increased to 1000 rpm. After at least another 90 minutes, the speed mixing device 603 is slowly increased to 1400 rpm. The speed of mixing device 603 is maintained at 1400 rpm, for between 12 hours and 72 hours, such as approximately 48 hours.

In STEP 1560, comprising an optional step, the initial pH of the digest can be recorded and/or adjusted to comprise a target pH. The target pH can be configured to improve the digest's shelf-life and/or solubility. In some embodiments, the target pH can comprise a pH greater than approximately 7.4. For example, approximately 54, of the digest is added to 0-3 pH paper and the pH is recorded, and approximately 54, of the digest is added to 1.0-12.0 pH paper and the pH is recorded. If the digest comprises an initial pH less than approximately 7.4, a basic solution (e.g. NaOH) can be added to the digest until a pH greater than approximately 7.4 is achieved.

In STEP 1570, comprising an optional step, the initial digest volume can be adjusted to comprise a target digest volume. If the digest comprises a volume less than the target digest volume, 0.01 N HCl is added to reach the target volume. In some embodiments, the target digest volume can comprise 900 mL.

In STEP 1580, comprising an optional step, one, two, or more excipients 711 can be added to the digest.

In STEP 1590, comprising an optional step, one, two, or more radioprotectants 712 can be added to the digest.

Upon the conclusion of Method 1500, ground ECM 120 comprises a digested extracellular matrix (referred to as “digested ECM 120” herein).

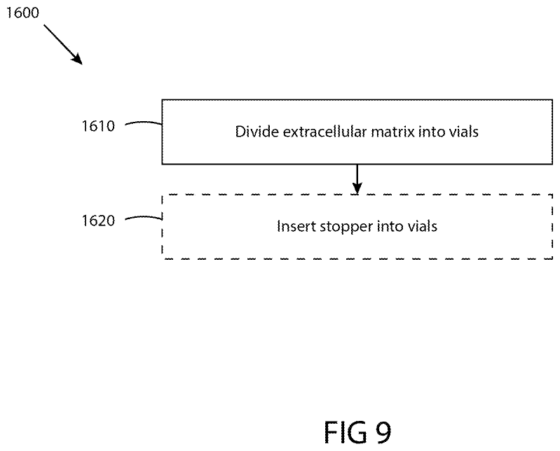

Referring now to FIG. 9, a method for aliquoting an extracellular matrix between one, two, or more containers is illustrated, consistent with the present inventive concepts. Method 1600 can be configured to aliquot digested ECM 120 produced in Method 1500 described herein in reference to FIG. 8, between one, two, or more containers. Method 1600 is configured to be performed within an environment suitable for aseptic processing, such that that materials, devices, and components utilized in Method 1600 are transferred to and/or contained within an environment suitable for aseptic processing. For example, Method 1600 is performed utilizing a sterile work area and/or sterile handling, such as to prevent or otherwise reduce contamination from microorganisms (e.g. bacteria, fungi, virus, etc.). As described herein, STEPs 1610 and 1620 are performed within the aseptic environment.

In STEP 1610, digested ECM 120 is aliquoted between one, two, or more vials 210 adhering to commonly known aseptic practices, such as to prevent contamination of digested ECM 120 from pathogens. In some embodiments, each vial 210 receives between 0.25 mL and 5 mL of digested ECM 120, such as 1±0.1 mL. Alternatively, digested ECM 120 can be aliquoted between one, two, or more syringes 220.

In some embodiments, the containers (e.g. vials 210, syringe 220) are sterilized prior to receiving digested ECM 120.

In some embodiments, digested ECM 120 is manually aliquoted via instrument 605 into vial 210. In some embodiments, digested ECM 120 is automatically aliquoted via a pump, such as a peristaltic pump, into vial 210.

In STEP 1620, comprising an optional step, a stopper 215 can be inserted into the opening of vial 210 comprising digested ECM 120. In some embodiments, stopper 215 further includes a fluid exchange element configured to allow for the passage of fluid between vial 210 and an external environment. In other embodiments, stopper 215 does not include a fluid exchange element and is configured to prevent or otherwise reduce the passage of fluid between vial 210 and an external environment.

Referring now to FIG. 10, a method for lyophilizing a container comprising an extracellular matrix is illustrated, consistent with the present inventive concepts. Method 1700 can be configured to lyophilize vials 210 comprising digested ECM 120 produced in Method 1600 described herein in reference to FIG. 9. Method 1700 is configured to be performed within an environment suitable for aseptic processing, such that that materials, devices, and components utilized in Method 1700 are transferred to and/or contained within an environment suitable for aseptic processing. For example, Method 1700 is performed utilizing a sterile work area and/or sterile handling, such as to prevent or otherwise reduce contamination from microorganisms (e.g. bacteria, fungi, virus, etc.). As described herein, STEPs 1710-1740 are performed within the aseptic environment.

In STEP 1710, one, two, or more vials 210 comprising digested ECM 120 from STEP 1620 are loaded into lyophilization device 606. In some embodiments, vials 210 are loaded into a preconditioned lyophilization device 606.

In STEP 1720, vials 210 comprising ECM 120 are lyophilized via lyophilization device 606. In some embodiments, lyophilization device 606 is configured to freeze vials 210 at a temperature of approximately −40° C. for no less than 4 hours. In some embodiments, lyophilization device 606 is configured to apply a vacuum source to vials 210. In some embodiments, the vacuum source comprises 150 micrometers of Hg. In some embodiments, lyophilization device 606 is configured to dry vials 210 at a temperature of between −8° C. and 0° C., increasing the temperature over time. In some embodiments, lyophilization device 606 is configured to increase the temperature to between 20° C. and 25° C., such as temperature of 22° C. (e.g. room temperature). In some embodiments, the total cycle duration comprises a duration of between 12 and 66 hours, such as a duration between 18 and 24 hours, such as approximately 24 hours.

In STEP 1730, comprising an optional step, an inert gas can be introduced into lyophilization device 606. In some embodiments, the inert gas comprises nitrogen.

In STEP 1740, vials 210 comprising digested ECM 120 are removed from lyophilization device 606.

In STEP 1750, comprising an optional step, a stopper 215 can be inserted into the opening of vial 210 comprising digested ECM 120, such as when a stopper 215 was not previously inserted into vials 210 during Method 2000.

In STEP 1760, comprising an optional step, a seal can be applied to surround at least the interface between vial 210 and stopper 215.

Upon the conclusion of Method 1700, digested ECM 120 comprises a lyophilized digested extracellular matrix (referred to as “lyophilized digested ECM 120” herein).

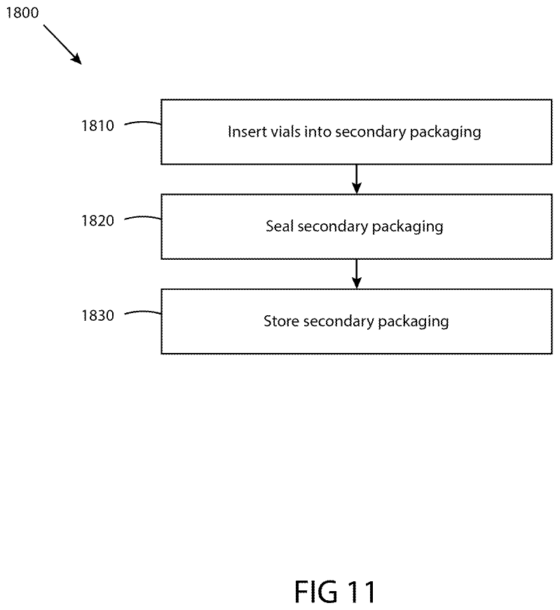

Referring now to FIG. 11, a method for packaging and storing a container comprising an extracellular matrix is illustrated, consistent with the present inventive concepts. Method 1800 can be configured to package vials 210 comprising lyophilized digested ECM 120 produced in Method 1700 described herein in reference to FIG. 10. Vials 210 can be packaged for bulk storage and/or sterilization. Method 1800 is configured to be performed within an environment suitable for aseptic processing, such that that materials, devices, and components utilized in Method 1800 are transferred to and/or contained within an environment suitable for aseptic processing. For example, Method 1800 is performed utilizing a sterile work area and/or sterile handling, such as to prevent or otherwise reduce contamination from microorganisms (e.g. bacteria, fungi, virus, etc.). As described herein, STEPs 1810-1820 are performed within the aseptic environment.

In STEP 1810, insert vials 210 from STEP 1730 or 1740 into one or more secondary packaging 611.

In STEP 1820, seal secondary packaging 611.

In STEP 1830, store secondary packaging 611 at a temperature of between 2° C. and 8° C., such as at a temperature of approximately 5° C.

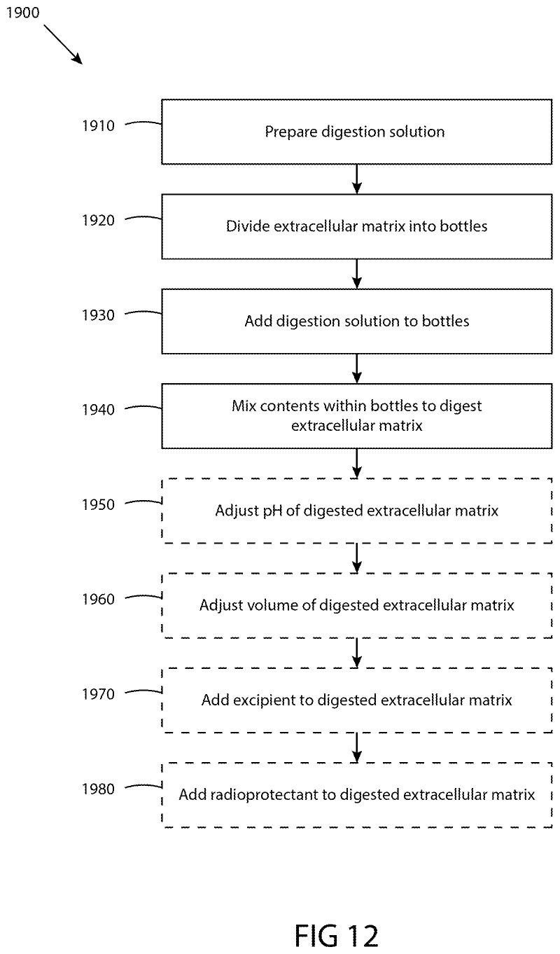

Referring now to FIG. 12, a method for digesting an extracellular matrix is illustrated, consistent with the present inventive concepts. Method 1900 can be configured to digest ground ECM 120 produced in Method 1400 described herein in reference to FIG. 7. In some embodiments, the materials, devices, and/or components utilized in Method 1900 comprise a pre-sterilized, semi-closed, and/or single-use system.

In STEP 1910, acid solution 709 is prepared and further combined with digestive enzyme 710 (collectively “digestion solution” herein).

In STEP 1920, ground ECM 120 is divided into one, two, or more bottles 610. In some embodiments, between 1 g and 20 g, such as 8.4 g, of ground ECM 120 is added to each bottle 610. In some embodiments, bottles 610 are sterilized prior to receiving ground ECM 120.

In STEP 1930, the digestion solution from STEP 1910 is added to each bottle 610 comprising ground ECM 120. In some embodiments, between 250 mL and 1000 mL, such as 900 mL, of the digestion solution is added to each bottle 610. In some embodiments, the collective volume of ground ECM 120 and the digestion solution comprises greater than or equal to 70% of the total volume of bottle 610. In some embodiments, the final concentration of ground ECM 120 in the digestion solution is between 0.5 mg/mL and 100 mg/mL, such as 10 mg/mL.

In STEP 1940, each bottle 610 comprising ground ECM 120 and the digestion solution (collectively “digest” herein) is stored in chamber 601 at a temperature of between 2° C. and 37° C., such as at a temperature between 15° C. and 30° C., such as at a temperature between 18° C. and 23° C., and for a duration of at least 12 hours, such as for a duration between 46 and 50 hours. Mixing device 603 is lowered into each bottle 610. Mixing device 603 is configured to stir the digest at a speed between 100 rpm and 5000 rpm, such as between 700 rpm and 1400 rpm. In some embodiments, mixing device 603 is positioned between ⅓ and ½ of the height of bottle 610, such as to promote, or otherwise improve, the homogeneity of the digestion process.

For example, the speed of mixing device 603 begins at 700 rpm. After at least 90 minutes, the speed of mixing device is slowly increased to 1000 rpm. After at least another 90 minutes, the speed mixing device 603 is slowly increased to 1400 rpm. The speed of mixing device 603 is maintained at 1400 rpm, for between 12 hours and 72 hours, such as approximately 48 hours.

In STEP 1950, comprising an optional step, the initial pH of the digest can be recorded and/or adjusted to comprise a target pH. The target pH can be configured to improve the digest's shelf-life and/or solubility. In some embodiments, the target pH can comprise a pH greater than approximately 7.4. For example, approximately 54, of the digest is added to 0-3 pH paper and the pH is recorded, and approximately 54, of the digest is added to 1.0-12.0 pH paper and the pH is recorded. If the digest comprises an initial pH less than approximately 7.4, a basic solution (e.g. NaOH) can be added to the digest until a pH greater than approximately 7.4 is achieved.

In STEP 1960, comprising an optional step, the digest volume can be adjusted to comprise a target digest volume. If the digest comprises a volume less than the target digest volume, 0.01 N HCl is added to reach the target volume. In some embodiments, the target digest volume can comprise 900 mL.

In STEP 1970, comprising an optional step, one, two, or more excipients 711 can be added to the digest.

In STEP 1980, comprising an optional step, one, two, or more radioprotectants 712 can be added to the digest.

Upon the conclusion of Method 1900, ground ECM 120 comprises a digested extracellular matrix (referred to as “digested ECM 120” herein).

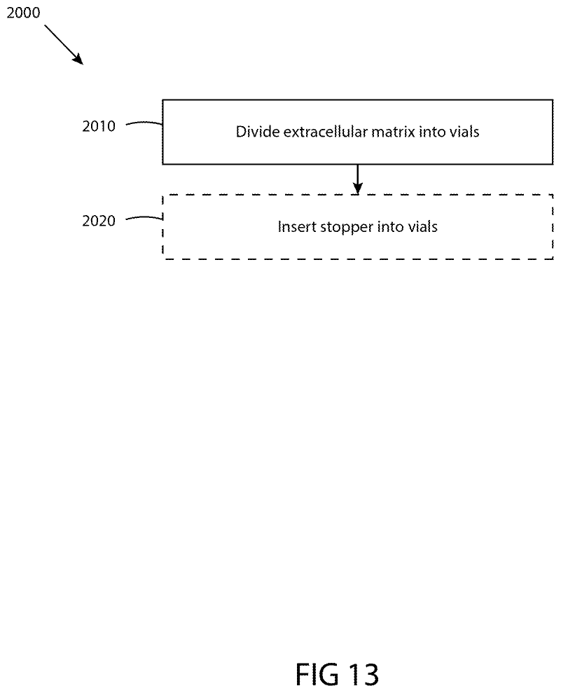

Referring now to FIG. 13, a method for aliquoting an extracellular matrix between one, two, or more containers is illustrated, consistent with the present inventive concepts. Method 2000 can be configured to aliquot digested ECM 120 produced in Method 1900 described herein in reference to FIG. 12, between one, two, or more containers.

In STEP 2010, digested ECM 120 is aliquoted between one, two, or more vials 210. In some embodiments, each vial 210 receives between 0.25 mL and 5 mL of digested ECM 120, such as 1±0.1 mL. Alternatively, digested ECM 120 can be aliquoted between one, two, or more syringes 220.

In some embodiments, the containers (e.g. vials 210, syringe 220) are sterilized prior to receiving digested ECM 120.

In some embodiments, digested ECM 120 is manually aliquoted via instrument 605 into vial 210. In some embodiments, digested ECM 120 is automatically aliquoted via a pump, such as a peristaltic pump, into vial 210.

In STEP 2020, comprising an optional step, a stopper 215 can be inserted into the opening of vial 210 comprising digested ECM 120. In some embodiments, stopper 215 further includes a fluid exchange element configured to allow for the passage of fluid between vial 210 and an external environment. In other embodiments, stopper 215 does not include a fluid exchange element and is configured to prevent or otherwise reduce the passage of fluid between vial 210 and an external environment.

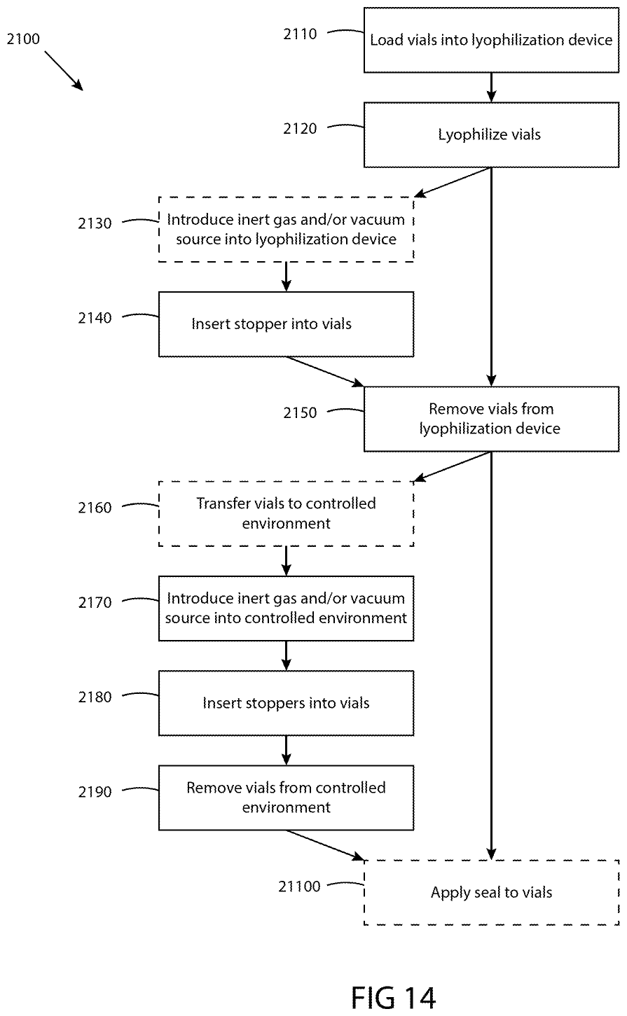

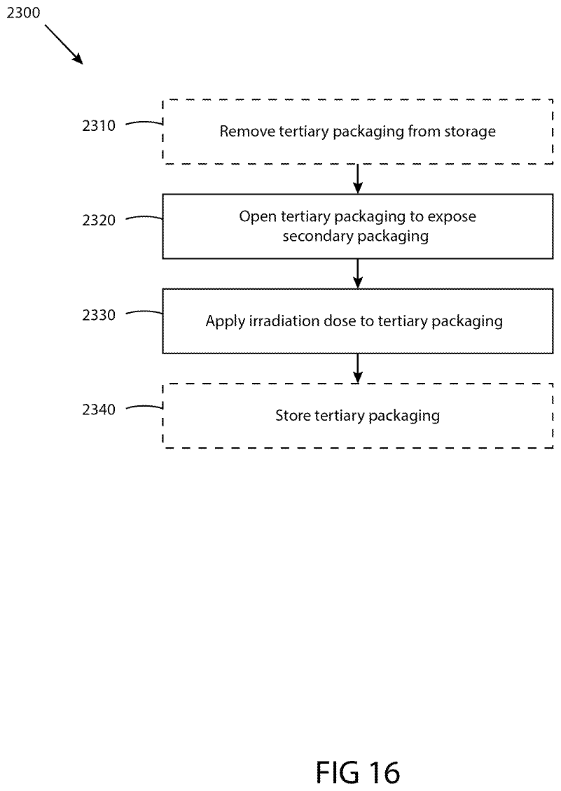

Referring now to FIG. 14, a method for lyophilizing a container comprising an extracellular matrix, is illustrated, consistent with the present inventive concepts. Method 2100 can be configured to lyophilize vials 210 comprising digested ECM 120 produced in Method 2000 described herein in reference to FIG. 13. In some embodiments, Method 1800 is configured to be performed prior to an irradiation based sterilization of vials 210 comprising digested ECM 120 as described herein in reference to FIG. 16.

In STEP 2110, one, two, or more vials 210 comprising digested ECM 120 from STEP 2020 are loaded into lyophilization device 606. In some embodiments, vials 210 are loaded into a preconditioned lyophilization device 606.

In STEP 2120, vials 210 comprising ECM 120 are lyophilized via lyophilization device 606. In some embodiments, lyophilization device 606 is configured to freeze vials 210 at a temperature of approximately −40° C. for no less than 4 hours. In some embodiments, lyophilization device 606 is configured to apply a vacuum source to vials 210. In some embodiments, the vacuum source comprises 150 micrometers of Hg. In some embodiments, lyophilization device 606 is configured to dry vials 210 at a temperature of between −8° C. and 0° C., increasing the temperature over time. In some embodiments, lyophilization device 606 is configured to increase the temperature to between 20° C. and 25° C., such as temperature of 22° C. (e.g. room temperature). Following STEP 2120, Method 2100 can proceed to STEP 2130 or STEP 2150 (e.g. STEPs 2130 and 2140 are not performed).

In STEP 2130, comprising an optional step, an inert gas and/or vacuum source can be introduced into lyophilization device 606. In some embodiments, the inert gas comprises nitrogen.

In STEP 2140, a stopper 215 is inserted into the opening of vial 210 comprising digested ECM 120. Insertion of stopper 215 can be configured to trap or otherwise maintain the inert gas and/or vacuum within vial 210. Stopper 215 can be inserted into the opening of vials 210 via an internal mechanism of lyophilization device 606. In some embodiments, stopper 215 is inserted into the opening of vial 210 prior to the release and/or removal of the inert gas and/or vacuum source from lyophilization device 606 (e.g. prior to the opening of lyophilization device 606).

In STEP 2150, vials 210 comprising digested ECM 120 are removed from lyophilization device 606. Following STEP 2120, Method 2100 can proceed to at least one of STEP 2160 or STEP 2100 (e.g. STEPs 2160-2190 are not performed).

In STEP 2160, comprising an optional step, vials 210 can be transferred to a controlled environment configured to maintain the sterility of vials 210 comprising digested ECM 120, such as a glove box or other airtight chamber.

In STEP 2170, an inert gas and/or vacuum source is introduced into the controlled environment. In some embodiments, the inert gas comprises nitrogen.

In STEP 2180, a stopper 215 is inserted into the opening of vial 210 comprising digested ECM 120, such as when stopper 215 was not previously inserted into vials 210 during Method 2000 (e.g. during optional step 2140). Insertion of stopper 215 can be configured to trap or otherwise maintain the inert gas and/or vacuum source within vial 210. In some embodiments, stopper 215 is inserted into the opening of vial 210 prior to the release and/or removal of the inert gas and/or vacuum source from the controlled environment.

In STEP 2190, vials 210 comprising digested ECM 120 are removed from the controlled environment.

In STEP 21100, comprising an optional step, a seal can be applied to surround at least the interface between vial 210 and stopper 215.

Upon the conclusion of Method 2100, digested ECM 120 comprises a lyophilized digested extracellular matrix (referred to as “lyophilized digested ECM 120” herein).

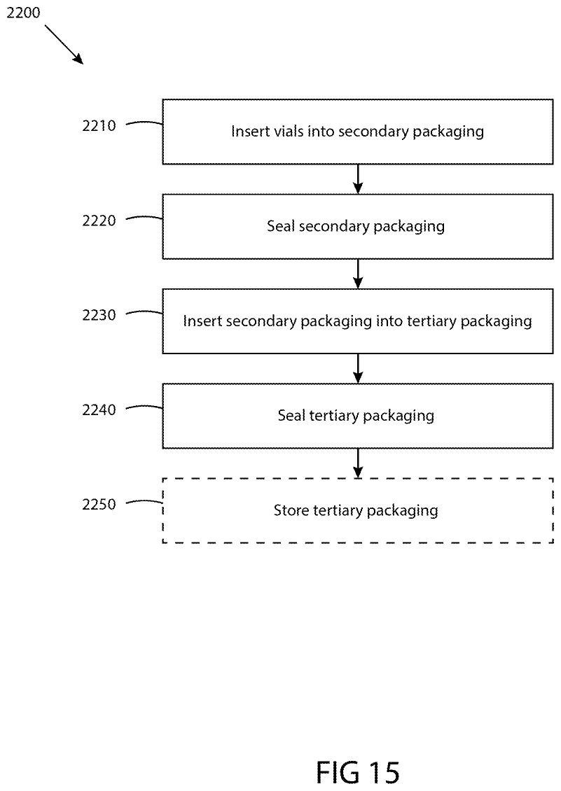

Referring now to FIG. 15, a method for packaging and storing a container comprising an extracellular matrix, and prior to an irradiation sterilization of the container comprising the extracellular matrix, is illustrated, consistent with the present inventive concepts. Method 2200 can be configured to package vials 210 comprising lyophilized digested ECM 120 produced in Method 2100 described herein in reference to FIG. 14. Vials 210 can be packaged for bulk storage and/or sterilization. In some embodiments, Method 1800 is configured to be performed prior to an irradiation based sterilization of vials 210 comprising digested ECM 120 as described herein in reference to FIG. 16.

In STEP 2210, insert vials 210 into one or more secondary packaging 611.

In STEP 2220, seal secondary packaging 611.

In STEP 2230, insert secondary packaging 611 into one or more tertiary packaging 612.

In STEP 2240, seal tertiary packaging 612.

In STEP 2250, comprising an optional step, tertiary packaging 612 comprising secondary packaging 611 can be stored at a temperature of between 2° C. and 8° C., such as at a temperature of approximately 5° C.

Referring now to FIG. 16, a method for an irradiation based sterilization of a container comprising an extracellular matrix is illustrated, consistent with the present inventive concepts. Method 2300 can be configured to sterilize (e.g. terminally sterilize) vials 210 comprising lyophilized digested ECM 120 produced in Method 2100 described herein in reference to FIG. 14 and/or packaged in Method 2200 described herein in reference to FIG. 15.

Vials 210 comprising lyophilized digested ECM 120 can be sterilized via gamma irradiation, such that vials 210 are exposed to gamma radiation (e.g. Cobalt 60). Vials 210 can be exposed to gamma radiation in doses ranging between 8 kGy and 25 kGy, such as a dose of 8 kGy, such as a dose of 12.5 kGy, such as a dose of 15 kGy. In some embodiments, vials 210 are sealed during the gamma radiation exposure.

Vials 210 comprising lyophilized digested ECM 120 can be sterilized via electron-beam irradiation (“e-beam irradiation” herein), such that vials 210 are exposed to beta radiation. Vials 210 can be exposed to beta radiation in doses ranging between 8 kGy and 25 kGy, such as a dose of 17.5 kGy. In some embodiments, vials 210 are sealed during the beta radiation exposure. Vials 210 can be treated to protect from ionizing radiation damage. In some embodiments, the irradiation dose is fractioned into multiple smaller doses. In some embodiments, lyophilized digested ECM 120 is kept at low temperature during irradiation. In some embodiments, lyophilized digested ECM 120 can be surrounded by an inert gas, such as nitrogen, thereby fully displacing the presence of oxygen in vial 210. In some embodiments, radioprotectant 712 is added to lyophilized digested ECM 120, such as to protect ECM 120 from radiation damage.

In STEP 2310, comprising an optional step, tertiary packaging 612 can be removed from storage.

In STEP 2320, open tertiary packaging 612 to expose secondary packaging 611. In some embodiments, secondary packaging 611 are further arranged in a predetermined spatial configuration within the tertiary container.

In STEP 2330, apply a predetermined irradiation dose to tertiary packaging 612 with secondary packaging 611 therein.

In STEP 2340, comprising an optional step, tertiary packaging 612 comprising secondary packaging 611 can be stored at a temperature of between 2° C. and 8° C., such as at a temperature of approximately 5° C.

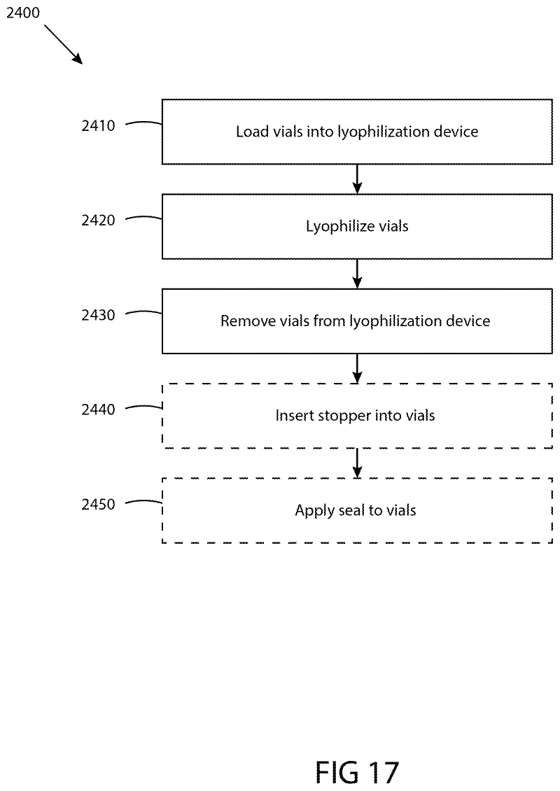

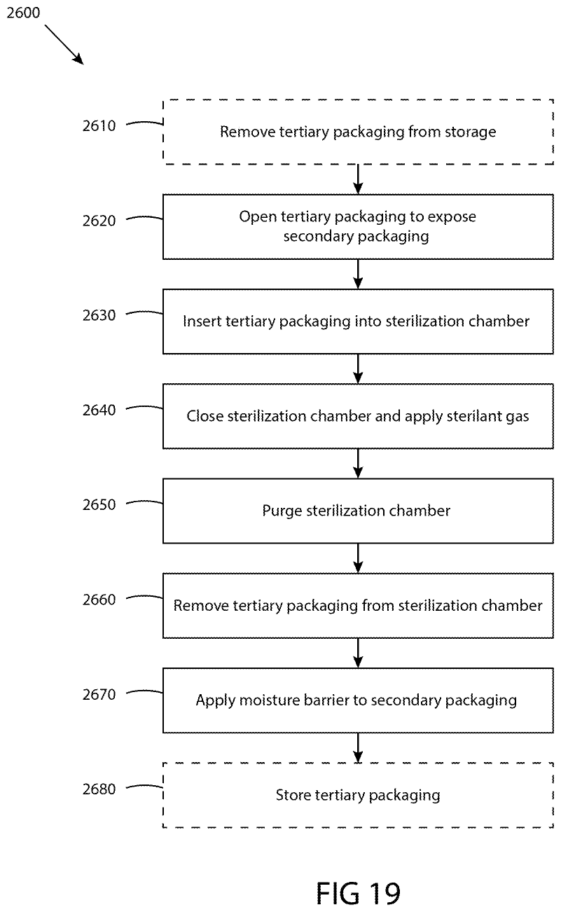

Referring now to FIG. 17, a method for lyophilizing a container comprising an extracellular matrix, is illustrated, consistent with the present inventive concepts. Method 2400 can be configured to lyophilize vials 210 comprising digested ECM 120 produced in Method 2000 described herein in reference to FIG. 13. In some embodiments, Method 2400 is configured to be performed prior to a gas based sterilization of vials 210 comprising digested ECM 120 as described herein in reference to FIG. 19.

In STEP 2410, one, two, or more vials 210 comprising digested ECM 120 from STEP 2020 are loaded into lyophilization device 606. In some embodiments, vials 210 are loaded into a preconditioned lyophilization device 606.

In STEP 2420, vials 210 comprising ECM 120 are lyophilized via lyophilization device 606. In some embodiments, lyophilization device 606 is configured to freeze vials 210 at a temperature of approximately −40° C. for no less than 4 hours. In some embodiments, lyophilization device 606 is configured to apply a vacuum source to vials 210. In some embodiments, the vacuum source comprises 150 micrometers of Hg. In some embodiments, lyophilization device 606 is configured to dry vials 210 at a temperature of between −8° C. and 0° C., increasing the temperature over time. In some embodiments, lyophilization device 606 is configured to increase the temperature to between 20° C. and 25° C., such as temperature of 22° C. (e.g. room temperature). In some embodiments, the total cycle duration comprises a duration of between 12 and 66 hours, such as a duration between 18 and 24 hours, such as approximately 24 hours.

In STEP 2430, vials 210 comprising digested ECM 120 are removed from lyophilization device 606.

In STEP 2440, comprising an optional step, a stopper 215 can be inserted into the opening of vial 210 comprising digested ECM 120, such as when a stopper 215 was not previously inserted into vials 210 during Method 2000. In some embodiment, stopper 215 further comprises a fluid exchange element. Alternatively or additionally, a seal can be applied to at least a portion of the opening of vial 210 comprising digested ECM 1290.

In STEP 2450, comprising an optional step, a seal can be applied to surround at least the interface between vial 210 and stopper 215.

Upon the conclusion of Method 2400, digested ECM 120 comprises a lyophilized digested extracellular matrix (referred to as “lyophilized digested ECM 120” herein).

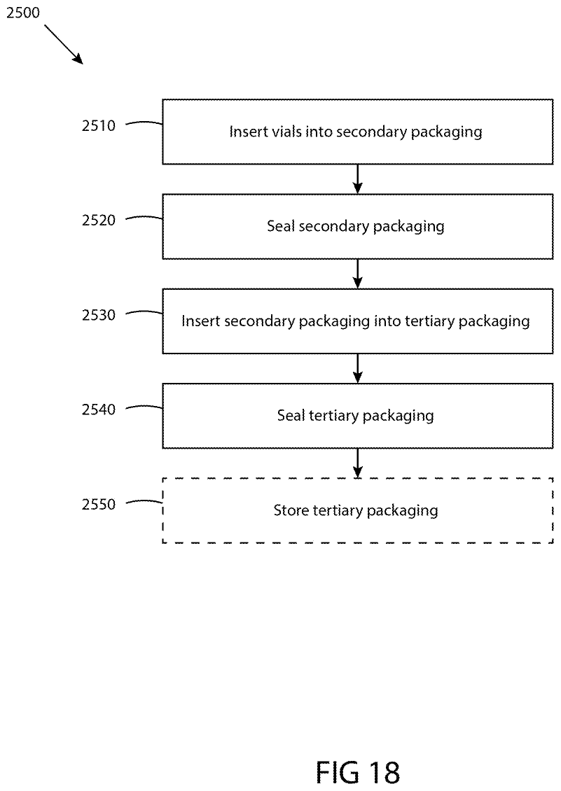

Referring now to FIG. 18, a method for packaging and storing a container comprising an extracellular matrix, consistent with the present inventive concepts. Method 2500 can be configured to package vials 210 comprising lyophilized digested ECM 120 produced in Method 2400 described herein in reference to FIG. 17. Vials 210 can be packaged for bulk storage and/or sterilization. In some embodiments, Method 2500 is configured to be performed prior to a gas based sterilization of vials 210 comprising digested ECM 120 as described herein in reference to FIG. 19.

In STEP 2510, insert vials 210 into one or more secondary packaging 611.

In STEP 2520, seal secondary packaging 611.

In STEP 2530, insert secondary packaging 611 into one or more tertiary packaging 612.

In STEP 2540, seal tertiary packaging 612.

In STEP 2550, comprising an optional step, tertiary packaging 612 comprising secondary packaging 611 can be stored at a temperature of between 2° C. and 8° C., such as at a temperature of approximately 5° C.

Referring now to FIG. 19, a method for sterilizing a container comprising an extracellular matrix is illustrated, consistent with the present inventive concepts. Method 2600 can be configured to sterilize (e.g. terminally sterilize) vials 210 comprising lyophilized digested ECM 120 produced in Method 2400 described herein in reference to FIG. 17 and/or packaged in Method 2500 described herein in reference to FIG. 18.

Vials 210 comprising lyophilized digested ECM 120 can be sterilized via supercritical carbon dioxide, such that vials 210 are exposed to sCO₂ in combination with peracetic acid. Super critical carbon dioxide sterilization including both “dry” and “wet” supercritical carbon dioxide on un-capped vials 210

Vials 210 comprising lyophilized digested ECM 120 can be sterilized via ethylene oxide, such that vials 210 are exposed to ethylene oxide gas within a chamber. The chamber can comprise a temperature of between 30° C. and 60° C., such as a temperature of between 30° C. and 50° C., and can comprise a relative humidity greater than or equal to 30%. Vials 210 can be exposed to ethylene oxide gas for a 16-hour cycle. In some embodiments, vials 210 are not sealed (e.g. un-capped) during the ethylene oxide gas exposure.

Vials 210 comprising lyophilized digested ECM 120 can be sterilized via vaporized peracetic acid, such that vials 210 are exposed to vaporized peracetic acid sterilization with high gas (23 mL, 4 injections=92 mL total), medium gas (15 mL, 6 injections=90 mL total), and low gas (20 mL, 2 injections=40 mL total).

Vials 210 comprising lyophilized digested ECM 120 can be sterilized via nitrogen dioxide, such that vials 210 are exposed to nitrogen dioxide sterilization with high gas (23 mL, 4 injections=92 mL total), medium gas (15 mL, 6 injections=90 mL total), and/or low gas (20 mL, 2 injections=40 mL total).

In STEP 2610, remove tertiary packaging 612 from storage.

In STEP 2620, open tertiary packaging 612 to expose secondary packaging 611.

In STEP 2630, insert tertiary packaging 612 with secondary packaging 611 therein into sterilization chamber 613.

In STEP 2640, close sterilization chamber 613 and apply a sterilant gas at defined pressure, sterilizing gas concentration, humidity, and/or time.

In STEP 2650, purge sterilization chamber 613 at a defined vacuum, temperature, humidity, and/or time.

In STEP 2660, remove tertiary packaging 612 from sterilization chamber 613.

In STEP 2670, apply a moisture barrier over-packaging to secondary packaging 611.

In STEP 2680, comprising an optional step, tertiary packaging 612 comprising secondary packaging 611 can be stored at a temperature of between 2° C. and 8° C., such as at a temperature of approximately 5° C.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for treating a patient comprising:
an extracellular matrix comprising tissue harvested from a tissue source;
a neutralizing element; and
a reconstituting element;
wherein the system is configured to provide a therapeutic benefit to the patient. reflected light collected by the optical assembly.

2.-218. (canceled)
